(19) United States
(12) Patent Application Publication
(10) Pub. No.: US 2015/0133467 A1
(43) Pub. Date: May 14, 2015

(54) SCREENING METHOD, PROTEIN INSTABILITY AND/OR STABILITY INDUCERS, AND PROTEIN ACTIVITY ASSESSMENT

(71) Applicants: KYOTO UNIVERSITY, Kyoto-shi, Kyoto (JP); NATIONAL UNIVERSITY CORPORATION TOKYO MEDICAL AND DENTAL UNIVERSITY, Tokyo (JP)

(72) Inventors: Masatoshi Hagiwara, Kyoto (JP); lsao Kii, Kyoto (JP); Takamitsu Hosoya, Tokyo (JP); Yuto Sumida, Tokyo (JP); Suguru Yoshida, Tokyo (JP)

(73) Assignee: KYOTO UNIVERSITY, Kyoto-shi, Kyoto (JP)

(21) Appl. No.: 14/406,101

(22) PCT Filed: Jun. 6, 2013
(86) PCT No.: PCT/JP2013/065723

§ 371 Date: Dec. 5, 2014

(30) Foreign Application Priority Data
Jun. 6, 2012 (JP) 2012-129094

(51) Int. Cl. A61K 31/496

(52) U.S. Cl. CPC A61K 31/496
USPC 514/253.13; 544/365; 435/375

(57) ABSTRACT

Provided is a screening method with which it is possible to determine the cause of a decrease or an increase in the amount of a target protein that is expressed by a cell. One or a plurality of embodiments include: conducting cultivation that includes bringing an assay cell into contact with a test substance, and measuring a relative amount (A) of the target protein in relation to an internal standard; conducting cultivation in which an assay cell is not brought into contact with a test substance, and measuring a relative amount (B) in relation to an internal standard; comparing the relative amount (A) and the relative amount (B); and, based on the comparison, selecting a candidate substance that induces instability and/or stability in the target protein. The assay cell is a cell that is able to express mRNA of the target protein and mRNA of the internal standard protein under the identical regulation of gene expression, or a cell having a means for expressing mRNA of the target protein and mRNA of the internal standard protein under the identical regulation of gene expression.

### TECHNICAL FIELD

The present disclosure relates to a method for screening a substance that induces instability and/or stability of protein; a compound that induces instability and/or stability of protein; a pharmaceutically acceptable salt thereof; a composition; use thereof; a method of inducing instability and/or stability of protein using the same; methods for prevention, improvement, suppression of progression and/or treatment of Alzheimer's disease or the like using the same; and, assessment of protein activity.

### BACKGROUND ART

Analysis of protein expression is performed by immunoassay, enzyme assay, RT-PCR, quantitative PCR, realtime PCR and the like. In addition to that, with improvement in cell imaging techniques, a highly automated cell analytical technique as a combination of fluorescence microscopy, quantitation of multi-parameter and image analysis, which is called a high-content screening (HCS), has been applied also to a protein expression analysis (see Non-patent documents 1-10).

### PRIOR ART DOCUMENTS

### Non-Patent Documents

### Patent Documents

[Non-patent document 1] Brodin P, Christophe T. High-content screening in infectious diseases. Curr Opin Chem Biol. 2011 August; 15(4):534-9.
[Non-patent document 2] Jain S, Heutink P. From single genes to gene networks: high-throughput-high-content screening for neurological disease. Neuron. 2010 Oct. 21; 68(2):207-17.
[Non-patent document 3] Daub A, Sharma P, Finkbeiner S. High-content screening of primary neurons: ready for prime time. Curr Opin Neurobiol. 2009 October; 19(5):537-43.
[Non-patent document 4] Bullen A. Microscopic imaging techniques for drug discovery. Nat Rev Drug Discov. 2008 January; 7(1):54-67.
[Non-patent document 5] Korn K, Krausz E. Cell-based high-content screening of small-molecule libraries. Curr Opin Chem Biol. 2007 October; 11(5):503-10.
[Non-patent document 6] Nicholson R L, Welch M, Ladlow M, Spring D R. Small-molecule screening: advances in microarraying and cell-imaging technologies. ACS Chem Biol. 2007 Jan. 23; 2(1):24-30.
[Non-patent document 7] Szymczak A L, Workman C J, Wang Y, Vignali K M, Dilioglou S, Vanin E F, Vignali D A. Correction of multi-gene deficiency in vivo using a single ‘self-cleaving’ 2A peptide-based retroviral vector. Nat Biotechnol. 2004 May; 22(5):589-94.
[Non-patent document 8] de Felipe P. Polycistronic viral vectors. Curr Gene Ther. 2002 September; 2(3):355-78.
[Non-patent document 9] Martínez-Salas E. Internal ribosome entry site biology and its use in expression vectors. Curr Opin Biotechnol. 1999 October; 10(5):458-64.
[Non-patent document 10] Houdebine L M, Attal J. Internal ribosome entry sites (IRESs) reality and use. Transgenic Res. 1999 June; 8(3):157-77.

### DISCLOSURE OF INVENTION

### Problem to be Solved by the Invention

There have been indications about relationships between overexpression or overactivation of various proteins and diseases. For example, reduction of a protein directed to such diseases is expected to result in therapies to these diseases.

Within a cell, stability of a protein is effected by various external factors. For example, a post-translational modification such as phosphorylation or acetylation is considered as one of the external factors. The post-translational modification is important for stability of a protein, and sometimes it is essential for functional expression of protein. Not a few human diseases are caused by overexpression or overactivation of proteins relating to diseases, and such an overexpression or overactivation is caused by abnormality in these external factors.

In general, screening (analysis) of candidate components of medical active ingredients is carried out in a test tube. On the other hand, when the target is an unknown molecular mechanism, a compound screening by use of cells is more favorable. However, in a case of a compound screening by use of cells, even when the amount of the target protein is reduced, it is difficult to determine at the stage of the screening whether the protein becomes instable or gene expression of the protein is suppressed.

In one or a plurality of embodiments, the present disclosure provides a screening method to allow determination whether increase or decrease of a target protein expressed by a cell is caused by enhancement or suppression of gene expression or a change in stability of the protein itself.

### Means for Solving Problem

In one or a plurality of embodiments, the present disclosure relates to a method for screening a substance that induces instability and/or stability of a target protein. In one or a plurality of embodiments, the screening method includes: conducting cultivation that includes bringing an assay cell into contact with a test substance, and measuring a relative amount (A) of the target protein expressed by the assay cell in relation to an internal standard protein expressed by the assay cell; culturing an assay cell without bringing it into contact with the test substance, and measuring a relative amount (B) of the target protein expressed by the assay cell in relation to the internal standard protein expressed by the assay cell; comparing the relative amount (A) and the relative amount (B); and on the basis of the comparison, selecting a candidate substance that induces instability and/or stability of the target protein. Here in one or a plurality of embodiments, the assay cell is a cell that can express mRNA of the target protein and mRNA of the internal standard protein under an identical regulation of gene expression, or a cell that has a means capable of expressing mRNA of the target protein and mRNA of the internal standard protein under an identical regulation of gene expression.

In one or a plurality of embodiments, the present disclosure relates to a kit for performing the screening method according to the present disclosure, and the kit includes an assay cell or a gene expression vector. In one or a plurality of embodiments, the assay cell is a cell capable of expressing mRNA of a target protein and mRNA of an internal standard protein under an identical regulation of gene expression, or a cell having a means capable of expressing mRNA of a target protein and mRNA of an internal standard protein under an identical regulation of gene expression. In one or a plurality of embodiments, the gene expression vector is a gene expression vector constituted and adapted so that a gene of an arbitrary target protein can be incorporated and an internal standard protein is incorporated in advance and that mRNA of the target protein and mRNA of the internal standard protein are expressed under an identical regulation of gene expression.

In one or a plurality of embodiments, the present disclosure relates to a compound represented by General formula (I) below or a pharmaceutically acceptable salt thereof.

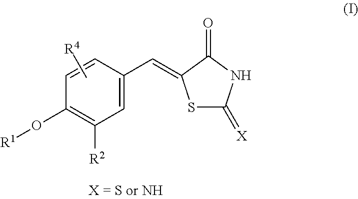

[In Formula (I), R¹ is either a hydrogen atom or a C₁₋₆ alkyl group; R² is selected from the group consisting of —R³, —C≡C—R³, —CH═CH—R³ and —O—(CH₂)ₙ—R³, where n is 1 to 6; R³ is selected from the group consisting of a hydrogen atom, a hydroxyl group, a C₁₋₈ alkyl group, —Si(R⁵)₃, and, a substituted or unsubstituted phenyl group, a monocyclic heteroaromatic group and a cyclic aliphatic group; or R¹ and R² are bonded to each other to form a ring, where —R¹-R²— is selected from the group consisting of —(CH₂)ₘ—CH₂—, —CH═CH—, —(CH₂)ₘ—O— and those substituted with halogen atoms, where m is 1 to 6; R⁴ is either a hydrogen atom or a C₁-6 alkyl group; and R⁵ is either a hydrogen atom or a C₁-6 alkyl group, where the three R⁵ in —Si(R⁵)₃ may be different from each other.]

In one or a plurality of embodiments, the present disclosure relates to a compound represented by General formula (III) below or a pharmaceutically acceptable salt thereof.

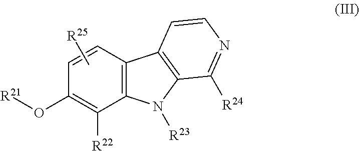

[In Formula (III), R²¹ and R²³ each independently is a hydrogen atom, a C₁₋₆ linear or branched or cyclic alkyl group, a benzyl or heteroaryl methyl group, a substituted or unsubstituted aryl group, or a substituted or unsubstituted heteroaryl group; R²² is selected from the group consisting of —R²⁶, —C≡C—R²⁶, —CH═CH—R²⁶ and —O—(CH₂)n-R²⁶, where n is 1 to 6; R²⁶ is selected from the group consisting of a hydrogen atom, a hydroxyl group, a C₁₋₈ alkyl group, —Si(R²⁷)₃, and, a substituted or unsubstituted phenyl group, a monocyclic heteroaromatic group and cyclic aliphatic group; or, R²¹ and R²² are bonded to each other to form a ring, —R²¹-R²²— is selected from the group consisting of —(CH₂)m-CH₂—, —CH═CH—, —(CH₂)m-O— and those substituted with a halogen atom, where m is 1 to 6; R²⁷ is a hydrogen atom, a C₁₋₆ alkyl group, a trihalomethyl group, or a hydroxyl group, and three R²⁷ in —Si(R²⁷)₃ may be different from each other; and R²⁴, R²⁵ are either hydrogen atoms or C₁₋₆ alkyl groups.]

In one or a plurality of embodiments, the present disclosure relates to a compound for inducing instability in an in vivo or intracellular DYRK1A protein or for reducing the amount of an in vivo or intracellular DYRK1A protein, a pharmaceutically acceptable salt thereof, or a composition including the same. In one or a plurality of embodiments, the present disclosure relates to a method for inducing instability in an in vivo or intracellular DYRK1A protein or for reducing the amount of an in vivo or intracellular DYRK1A protein.

In one or a plurality of embodiments, the present disclosure relates to a method for prevention, improvement, suppression of progression, and/or treatment of Alzheimer's disease by use of a compound for inducing instability in an in vivo or intracellular DYRK1A protein or for reducing the amount of an in vivo or intracellular DYRK1A protein, a pharmaceutically acceptable salt thereof, or a composition including the same.

In one or a plurality of embodiments, the present disclosure relates to a compound represented by General formula (II) below, a pharmaceutically acceptable salt thereof, or a composition including the same for inducing instability in an in vivo or intracellular TAU protein or for reducing the amount of an in vivo or intracellular TAU protein. In one or a plurality of embodiments, the present disclosure relates to a method for inducing instability in an in vivo or intracellular TAU protein or for reducing the amount of an in vivo or intracellular TAU protein.

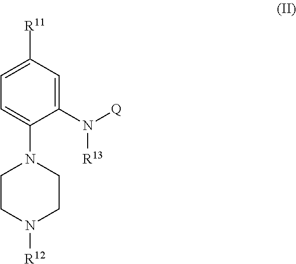

[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

In one or a plurality of embodiments, the present disclosure relates to a method for prevention, improvement, suppression of progression, and/or treatment of Alzheimer's disease or Tauopathies by use of a compound represented by General formula (II) below, a pharmaceutically acceptable salt thereof, or a composition including the same for inducing instability in an in vivo or intracellular TAU protein or for reducing the amount of an in vivo or intracellular TAU protein.

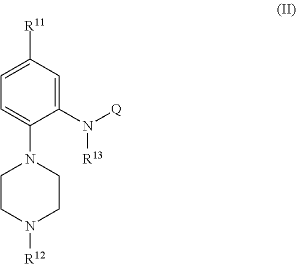

[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

In one or a plurality of embodiments, the present disclosure relates to use of a homocysteine concentration in blood as an index of in vivo DYRK1A protein activity.

### BRIEF DESCRIPTION OF DRAWINGS

FIG. 1 is a model diagram showing an embodiment of a simultaneous expression system of FLAG-tagged DYRK1A and EGFP (FLAG-DYRK1A-2A-EGFP).

FIG. 2 shows an example of a Western blot analysis indicating that expression induction of FLAG-DYRK1A and EGFP is conducted by doxycycline.

FIG. 3 shows an example of a Western blot analysis indicating that a test compound (Compound 1) does not affect the amount of EGFP protein of internal standard but reduces only the amount of FLAG-DYRK1A protein within the cell.

FIG. 4 shows an example of a result of Western blot analysis of the amount of protein of various phosphoenzymes at the time of adding 0, 4 and 8 μM of the Compound 1.

FIG. 5 is a model diagram of an embodiment of simultaneous expression system of FLAG-tagged DYRK1A and EGFP (FLAG-DYRK1A-2A-EGFP).

FIG. 6 shows an example of a Western blot analysis indicating that a test compound (Compound 2) does not affect the amount of mCherry protein of internal standard but reduces only the amount of EGFP-TAU protein within the cell.

FIG. 7 shows an example of a result of measurement of homocysteine concentration in blood in a case of oral administration of a DYRK1A inhibitor Harmine to rats.

FIG. 8 shows an example of a result of Western blot analysis of the amounts of DYRK1A protein at the time of adding 0 and 4 μM of Compounds 3, 4 or 5.

FIG. 9 shows an example of Western blot analysis indicating that a test compound (Compound 6) reduces the amount of FLAG-DYRK1A protein within the cell.

FIG. 10 The left of FIG. 10 shows an example of Western blot analysis indicating that a test compound (Compound 7) does not affect the amount of EGFP protein of internal standard but reduces only the amount of FLAG-DYRK1A protein within the cell. The right of FIG. 10 shows an example of Western blot analysis indicating that a test compound (Compound 8) does not affect the amount of EGFP protein of internal standard but reduces only the amount of FLAG-DYRK1A protein within the cell.

FIG. 11 shows an example of Western blot analysis indicating that a test compound (Compound 9) does not affect the amount of EGFP protein of internal standard but reduces only the amount of FLAG-DYRK1A protein within the cell.

FIG. 12 shows an example of Western blot analysis indicating that a test compound (Compound 10) reduces the amount of FLAG-DYRK1A protein within the cell.

### DESCRIPTION OF THE INVENTION

### [Screening Method]

In one or a plurality of embodiments, the present disclosure relates to a method for screening a substance that induces instability and/or stability in a target protein. In one or a plurality of embodiments, the screening method includes: conducting cultivation that includes bringing an assay cell into contact with a test substance, and measuring a relative amount (A) of the target protein expressed by the assay cell in relation to an internal standard protein expressed by the assay cell; culturing an assay cell without bringing it into contact with the test substance, and measuring a relative amount (B) of the target protein expressed by the assay cell in relation to the internal standard protein expressed by the assay cell; comparing the relative amount (A) and the relative amount (B); and on the basis of the comparison, selecting a candidate substance that induces instability and/or stability of the target protein.

In one or a plurality of embodiments, the screening method according to the present disclosure can be applied to living cells, or tissues, organs, or a living body. Although it is difficult to find out some molecular mechanisms by an in vitro analysis, the present embodiment can target such mechanisms and thus it is useful. For example, since most of the molecular mechanisms to ensure the stability of proteins relating to diseases have not been clarified yet, a screening based on living cells or the like is advantageous for the purpose of targeting such an unknown molecular mechanism.

### [Assay Cell]

In one or a plurality of embodiments, an assay cell in the screening method according to the present disclosure expresses a target protein and an internal standard protein. For example, even when the amount of a target protein is reduced in a screening method based on living cells or the like, in some cases it may be difficult to distinguish clearly at the stage of screening whether instability occurs or a gene expression is suppressed. If the assay cell expresses the internal standard protein, in a case where the amount of the target protein expressed by the assay cell is increased or decreased, it is possible to distinguish whether the increase/decrease is caused by enhancement or suppression of gene expression or by any change in stability of the protein itself.

In one or a plurality of embodiments, the assay cell in the screening method according to the present disclosure is a cell capable of expressing mRNA of a target protein and mRNA of an internal standard protein under an identical regulation of gene expression. Expressing mRNA of the target protein and mRNA of the internal standard protein under an identical regulation of gene expression is favorable since in a case where the amount of the target protein expressed by the assay cell is increased or decreased, it is possible to distinguish more clearly whether the increase/decrease is caused by enhancement or suppression of gene expression or by any change in stability of the protein itself. Or the possibility that the instability of the target protein comes from a secondary influence by cell damage or the like can be eliminated. In one or a plurality of embodiments, a cell that can express mRNA of a target protein and mRNA of an internal standard protein under an identical regulation of gene expression is a cell capable of expressing the target protein and the internal standard protein by an identical promoter. In one or a plurality of embodiments, a cell that can express mRNA of the target protein and mRNA of the internal standard protein by an identical promoter is a cell capable of expressing the target protein and the internal standard protein by a polycistronic gene expression system. In one or a plurality of embodiments, the polycistronic gene expression system is a gene expression system including a constitution where the target protein gene and the internal standard protein gene are connected to each other via an IRES sequence or a gene sequence that codes a self-cleavage peptide.

In one or a plurality of embodiments, the assay cell in the screening method according to the present disclosure is a cell having a means capable of expressing mRNA of a target protein and mRNA of an internal standard protein under an identical regulation of gene expression. The means capable of expressing mRNA of the target protein and mRNA of the internal standard protein under an identical regulation of gene expression is favorable since in a case where the amount of the target protein expressed by the assay cell is increased or decreased, it is possible to distinguish more clearly whether the increase/decrease is caused by enhancement or suppression of the gene expression or by any change in stability of the protein itself. Or a possibility that the instability of the target protein caused by a secondary influence of cell damage or the like can be eliminated. In one or a plurality of embodiments, the means capable of expressing mRNA of the target protein and mRNA of the internal standard protein under an identical regulation of gene expression is an expression system that expresses the target protein and the internal standard protein by an identical promoter. In one or a plurality of embodiments, the means capable of expressing mRNA of the target protein and mRNA of the internal standard protein under an identical regulation of gene expression is a polycistronic gene expression system where the target protein and the internal standard protein can be expressed with the identical mRNA. In one or a plurality of embodiments, the polycistronic gene expression system is a gene expression system including a constitution where a target protein gene and an internal standard protein gene are connected to each other via an IRES sequence or a gene sequence that codes a self-cleavage peptide.

For the “promoter” in the present disclosure, any promoter that has been known or will be progressed in the future can be used as long as it can induce expression of protein within an assay cell. For example, it may be a promoter like a CMV promoter (though not limited to this example) capable of constitutional expression. Alternatively, it may be a promoter such as a tetracycline expression induction system (though not limited to this example) that is capable of controlling ON/OFF of expression.

In one or a plurality of embodiments, though there is no particular limitation, the number of genes expressed as the identical mRNA in the “polycistronic gene expression system” in the present disclosure is 2, 3 or 4. In the present disclosure, “Internal Ribosome Entry Site: IRES sequence” is a sequence for recruiting ribosome on mRNA in a manner non-independent on a cap structure for allowing to start translation, and thus any IRES sequences that have been known or that will be progressed in the future can be applied. In one or a plurality of embodiments, though “self-cleavage peptide” in the present disclosure is derived from 2A gene of foot-and-mouth disease virus, the present disclosure is not limited to this and any self-cleavage peptide that has been known or that will be progressed in the future can be applied.

In one or a plurality of embodiments, an assay cell can be produced by introducing to a cell a gene expression vector that is constituted and adapted so that mRNA of a target protein and mRNA of an internal standard protein may be expressed under the identical regulation of gene expression. The cells used for production of the assay cell, namely, the cells to be the target for introduction of the vector are not limited in particular, and in one or a plurality of embodiments, they are cells of mammals. In one or a plurality of embodiments, the cells of mammals are the cells of human, bovine, cat, monkey, dog, elephant, hamster, mink, mouse, swine, rabbit, and rat. Though there is no particular limitation on the cell type, in one or a plurality of embodiments, the examples include: nerve cells or cultured cells thereof; hemocytometer cells or culture cells thereof; myeloid cells or culture cells thereof; epithelial cells or culture cells thereof; connective tissue cells or culture cells thereof; embryonic cells or culture cells thereof; cells derived from kidney or culture cells thereof; cells derived from liver or culture cells thereof; cells derived from lung or culture cells thereof; cells derived from brain or culture cells thereof; cells derived from a mammary gland or culture cells thereof; cells derived from bone or culture cells thereof; and cells derived from stomach or culture cells thereof. The vector may be a transient expression type or stable expression type.

### [Test Substance]

The test substances in the screening method according to the present disclosure are not limited in particular. In one or a plurality of embodiments in the present disclosure, the “substance” may be a compound, a composition, a mixture, an extract, a natural product, or a synthetic product. In one or a plurality of embodiments, a screening library can be used for the test substance, and though there is no particular limitation, libraries of compounds or the salts thereof, compositions, mixtures, extracts, natural products and synthetic products can be utilized.

In one or a plurality of embodiments, a contact between an assay cell and the test substance can be performed by culturing the assay cell in the presence of test substance though the present disclosure is not limited thereto. Further in one or a plurality of embodiments, the conditions for culturing the assay cell can be selected suitably in accordance with the kinds of the assay cell, but the present disclosure is not limited thereto. The contact time and the concentration of the test substance in the contact between the assay cell and the test substance can be determined suitably without any particular limitations.

### [Relative Amount]

In one or a plurality of embodiments, the relative amount in the screening method according to the present disclosure indicates the protein amount of the target protein in relation to the protein amount of the internal standard protein. The relative amount can be measured by any measurement method that has been known or that will be progressed in the future, without any particular limitations. In one or a plurality of embodiments, measurement of the relative amount is performed by optical imaging. Employment of the optical imaging is advantageous since it allows a high-speed screening and high throughput. In one or a plurality of embodiments, measurement of optical imaging is performed by observing the assay cell with a microscope thereby measuring fluorescence or luminescence from the target protein, and fluorescence or luminescence from the internal standard protein. In one or a plurality of embodiments, the means for measuring florescence or luminescence from the assay cell is a fluorescent or luminescent imaging apparatus equipped with a microscope, and in one or a plurality of embodiments, the apparatus includes further analysis software or an analyzer. In one or a plurality of embodiments, the means for obtaining fluorescence or luminescence from the target protein is labeling the target protein immunologically so as to emit fluorescence or luminescence, and in one or a plurality of embodiments, it is to allow the target protein to fuse with a tag. In one or a plurality of embodiments, the means to obtain fluorescence or luminescence from the internal standard protein is to use a fluorescent protein as the internal standard protein, that is, in one or a plurality of embodiments, it is to label the internal standard protein immunologically so as to emit fluorescence or luminescence, and in one or a plurality of embodiments, it is to allow the internal standard protein to fuse with a tag.

In one or a plurality of embodiments, “labeling immunologically so as to emit fluorescence” in the present disclosure includes a fluorescent cell staining detection method using an antibody bonded to a fluorescent protein. In one or a plurality of embodiments, “labeling immunologically so as to emit luminescence” in the present disclosure includes a chemiluminescence detection method using alkaline phosphatase labeled antibody for example (though it is not limited to this example). In the present disclosure, for the “tag”, any tag that has been known or that will be progressed in the future to be used for measurement of protein can be used, and in one or a plurality of embodiments, it is a fluorescent protein, and in one or a plurality of embodiments, it is an epitope tag such as a FLAG tag or HA tag (though it is not limited to these examples).

In one or a plurality of embodiments in the screening method according to the present disclosure, the target protein and the internal standard protein are expressed in an assay cell in a form enabling emission of fluorescence. This results in an advantage that the relative amount of the target protein can be observed in living cells.

### [Target Protein]

In the screening method according to the present disclosure, the “target protein” is not limited in particular. In one or a plurality of embodiments, the target protein may be for example the proteins such as DARK1A and TAU (but not limited thereto) that relate or are considered as relating to diseases. Overproduction of a phosphoenzyme DYRK1A is shown in Down's syndrome, and this overproduction is considered as causing Alzheimer's disease that develops with high probability in the Down's syndrome. Further, microtubule connected protein TAU is insolubilized and accumulated due to the over-phosphorylation and the accumulation of this over-phosphorylated TAU is considered as causing a neurodegeneration disease. The former and conventional analyses using gene-deleted mice indicate that it is possible to suppress development of Alzheimer's disease by deleting the TAU gene. This result implies that the development of Alzheimer's disease can be suppressed by reducing the TAU gene product (TAU protein).

In one or a plurality of embodiments, the target protein is expressed in the assay cell in a form being fused with a tag, or expressed in an assay cell in a form capable of emitting fluorescence. As mentioned above, these forms are advantageous in measuring the relative amount of the target protein by optical imaging.

### [Internal Standard Protein]

In the screening method according to the present disclosure, “internal standard protein” is not limited in particular. In one or a plurality of embodiments, the internal standard protein is a fluorescent protein such as GFP or EGFP for example (though it is not limited thereto). As mentioned above, this form is advantageous in measuring the relative amount of the target protein by optical imaging.

### [Comparison Between Relative Amounts (A) and (B); and Selection of Candidate Substances]

The screening method according to the present disclosure includes: conducting cultivation that includes bringing an assay cell into contact with a test substance, and measuring a relative amount (A) of the target protein expressed by the assay cell in relation to an internal standard protein expressed by the assay cell; culturing an assay cell without bringing the cell into contact with the test substance, and measuring a relative amount (B) of the target protein expressed by the assay cell in relation to the internal standard protein expressed by the assay cell; comparing the relative amount (A) and the relative amount (B); and selecting a candidate substance that induces the instability and/or stability of the target protein. In the screening method according to the present disclosure, there is no particular limitation on the method for selecting the candidate substance. In one or a plurality of embodiments, selection of the candidate substance includes selecting the test substance as a candidate substance for inducing instability of the target protein if the relative amount (A) is decreased in comparison with the relative amount (B); and/or selecting the test substance as a candidate substance for inducing stability of the target protein if the relative amount (A) is increased in comparison with the relative amount (B). In one or a plurality of embodiments, the selected candidate substance may be reviewed further to be determined as a substance to induce instability and/or stability of the target protein. In one or a plurality of embodiments, the selected candidate substance itself may be determined as a substance to induce instability and/or stability of the target protein.

### [Kit]

In one or a plurality of embodiments, the present disclosure relates to a kit for conducting the screening method according to the present disclosure. The kit according to the present disclosure includes an assay cell used for the screening method according to the present disclosure, or a gene expression vector for producing the assay cell. In one or a plurality of embodiments, the kit according to the present disclosure may include at least one selected from the group consisting of a medium and a reagent necessary for culturing an assay cell, a reagent necessary for producing the assay cell, polynucleotide, and an instruction manual for the assay cell or for the gene expression vector.

### [Gene Expression Vector]

In one or a plurality of embodiments, a gene expression vector included in the kit according to the present disclosure is a gene expression vector constituted and adapted so that the mRNA of the target protein and mRNA of the internal standard protein will be expressed under an identical regulation of gene expression. The vector is used for producing an assay cell. In one or a plurality of embodiments, in the vector, the expression regulation mechanism may be selected suitably in accordance with the kinds of the assay cell. In one or a plurality of embodiments, the vector may conduct a transient expression or may conduct a stable expression. In one or a plurality of embodiments, the gene expression vector included in the kit according to the present disclosure is the gene expression vector that can express the target protein and the internal standard protein by an identical promoter. In one or a plurality of embodiments, the gene expression vector that can express the target protein and the internal standard protein by an identical promoter is the gene expression vector that can express the target protein and the internal standard protein by a polycistronic gene expression system. In one or a plurality of embodiments, the polycistronic gene expression system is a gene expression system including a constitution where a target protein gene and an internal standard protein gene are connected via an IRES sequence or a gene sequence that codes the self-cleavage peptide.

Namely, the present disclosure may relate to the one or a plurality of embodiments.

[S1] A method for screening a substance that induces instability and/or stability of a target protein, the method comprising: conducting cultivation that comprises bringing an assay cell into contact with a test substance, and measuring a relative amount (A) of the target protein expressed by the assay cell in relation to an internal standard protein expressed by the assay cell; culturing an assay cell without bringing the cell into contact with the test substance, and measuring a relative amount (B) of the target protein expressed by the assay cell in relation to the internal standard protein expressed by the assay cell; comparing the relative amount (A) and the relative amount (B); and based on the comparison, selecting a candidate substance that induces the instability and/or stability of the target protein. In one or a plurality of embodiments, the assay cell is a cell capable of expressing mRNA of the target protein and mRNA of the internal standard protein under an identical regulation of gene expression, or a cell having a means capable of expressing mRNA of the target protein and mRNA of the internal standard protein under an identical regulation of gene expression.
[S2] The screening method according to [S1], wherein the selection of the candidate substance comprises, in a case where the relative amount (A) is decreased more than the relative amount (B), selecting the test substance as a candidate substance to induce instability of the target protein, and/or in a case where the relative amount (A) is increased more than the relative amount (B), selecting the test substance as a candidate substance to induce stability of the target protein.
[S3] The screening method according to [S1] or [S2], wherein the assay cell is a cell capable of expressing the target protein and the internal standard protein by an identical promoter.
[S4] The screening method according to [S3], wherein the assay cell is a cell capable of expressing the target protein and the internal standard protein by a polycistronic gene expression system.
[S5] The screening method according to [S4], wherein the polycistronic gene expression system comprises a constitution where a target protein gene and an internal standard protein gene are connected via either an IRES sequence or a gene sequence that codes a self cleavage peptide.
[S6] The screening method according to any one of [S1] to [S5], wherein at least either the target protein or the internal standard protein is expressed in the assay cell in the form of being fused with a tag.
[S7] The screening method according to any one of [S1] to [S6], wherein the target protein and the internal standard protein are expressed in the assay cell in the form capable of emitting fluorescence.
[S8] A kit for conducting the screening method according to any one of [S1] to [S7], comprising:

an assay cell capable of expressing mRNA of the target protein and mRNA of the internal standard protein under an identical regulation of gene expression, or an assay cell having a means capable of expressing mRNA of the target protein and mRNA of the internal standard protein under an identical regulation of gene expression; or

a gene expression vector having the gene of the internal standard protein, constituted and adjusted to allow incorporation of a gene of an arbitrary target protein and to allow expression of mRNA of the target protein and mRNA of the internal standard protein under an identical regulation of gene expression.

### [Substance Relating to Induction of Stability or Instability in DYRK1A]

In one or a plurality of embodiments, the present disclosure relates to a compound represented by General formula (I) below or a pharmaceutically acceptable salt thereof:

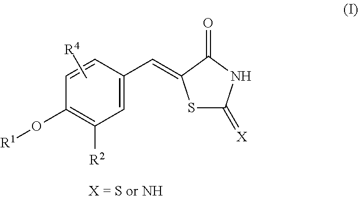

[In Formula (I), R¹ is either a hydrogen atom or a C₁₋₆ alkyl group; R² is selected from the group consisting of —R³, —C≡C—R³, —CH═CH—R³ and —O—(CH₂)ₙ—R³, where n is 1 to 6; R³ is selected from the group consisting of a hydrogen atom, a hydroxyl group, a C₁₋₈ alkyl group, —Si(R⁵)₃, and, a substituted or unsubstituted phenyl group, a monocyclic heteroaromatic group and a cyclic aliphatic group; or R¹ and R² are bonded to each other to form a ring, where —R¹-R²— is selected from the group consisting of —(CH₂)ₘ—CH₂—, —CH═CH—, —(CH₂)ₘ—O— and those substituted with halogen atoms, where m is 1 to 6; R⁴ is either a hydrogen atom or a C₁₋₆ alkyl group; and R⁵ is either a hydrogen atom or a C₁₋₆ alkyl group, where the three R⁵ in —Si(R⁵)₃ may be different from each other.]

In one or a plurality of embodiments, the present disclosure relates to a compound represented by General formula (III) below or a pharmaceutically acceptable salt thereof:

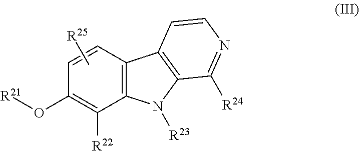

[In Formula (III), R²¹ and R²³ each independently is a hydrogen atom, a C₁₋₆ linear or branched or cyclic alkyl group, a benzyl or heteroaryl methyl group, a substituted or unsubstituted aryl group, or a substituted or unsubstituted heteroaryl group; R²² is selected from the group consisting of —R²⁶, —C≡C—R²⁶, —CH═CH—R²⁶ and —O—(CH₂)n-R²⁶, where n is 1 to 6; R²⁶ is selected from the group consisting of a hydrogen atom, a hydroxyl group, a C₁₋₈ alkyl group, —Si(R²⁷)₃, and, a substituted or unsubstituted phenyl group, a monocyclic heteroaromatic group and cyclic aliphatic group; or, R²¹ and R²² are bonded to each other to form a ring, —R²¹-R²²— is selected from the group consisting of —(CH₂)m-CH₂—, —CH═CH—, —(CH₂)m-O— and those substituted with a halogen atom, where m is 1 to 6; R²⁷ is a hydrogen atom, a C₁₋₆ alkyl group, a trihalomethyl group, or a hydroxyl group, and three R²⁷ in —Si(R²⁷)₃ may be different from each other; and R²⁴, R²⁵ are either hydrogen atoms or C₁₋₆ alkyl groups.]

In one or a plurality of embodiments, in the Formulae (I) and (III), “C₁₋₆ alkyl group” is a linear, branched or cyclic alkyl group having a carbon number in the range of 1 to 6. In one or a plurality of embodiments, examples of the linear or branched alkyl group having a carbon number of 1 to 6 include: a methyl group, an ethyl group, a 1-propyl group, a 2-propyl group, a 2-methyl-1-propyl group, a 2-methyl-2-propyl group, a 1-butyl group, a 2-butyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, and a 2,3-dimethyl-2-butyl group. Further, in one or a plurality of embodiments, examples of a cyclic alkyl group having a carbon number of 1 to 6 include cyclopropyl, cyclobutyl, a cyclopentyl, and cyclohexyl.

In Formula (I), “C₁₋₃ alkyl group” indicates a linear or branched alkyl group having a carbon number of 1 to 3, which is a monovalent group induced by subtracting one arbitrary hydrogen atom from an aliphatic hydrocarbon having a carbon number of 1 to 3, and the specific examples include a methyl group, an ethyl group, a 1-propyl group, and a 2-propyl group.

In Formula (I), “C₁₋₆ alkoxy group” indicates an oxy group in which the above-defined “C₁₋₆ alkyl group” is bound, and the specific examples include: a methoxy group, an ethoxy group, a 1-propyloxy group, a 2-propyloxy group, a 2-methyl-1-propyloxy group, a 2-methyl-2-propyloxy group, a 1-butyloxy group, a 2-butyloxy group, a 1-pentyloxy group, a 2-pentyloxy group, a 3-pentyloxy group, a 2-methyl-1-butyloxy group, a 3-methyl-1-butyloxy group, a 2-methyl-2-butyloxy group, a 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, and a 2,3-dimethyl-2-butyloxy group.

In Formulae (I) and (III), “heterocycle” indicates a non-aromatic ring or aromatic ring that contains one or two hetero atom(s) in atoms that constitute a ring, and the ring may include a double bond. In the present disclosure, “heteroaromatic ring” indicates an aromatic heterocycle. In the present disclosure, “hetero atom” indicates a sulfur atom, an oxygen atom or a nitrogen atom. Further in the present disclosure, “nitrogen-containing heterocycle” indicates a non-aromatic ring or an aromatic ring that contains one or two nitrogen atom(s) in atoms constituting a ring, and the ring may include a double bond.

In Formula (I) and (III), a “cyclic aliphatic group” indicates an aliphatic group having a cyclic structure. An example of the cyclic aliphatic group may be a cyclic aliphatic group having a carbon number of 3 to 10, and it may be a cyclic aliphatic group having an annelation structure constituted of plural rings. The specific examples include a cycloalkyl group, a cyclic ether group, a decahydronaphthyl group and an adamantyl group, each having a carbon number of 3 to 10. Specific examples of the cycloaliphatic group having a carbon number of 3 to 10 include a cyclopropyl group, a cyclobutyl group, a cyclopentyl group, a cyclohexyl group, and a cycloheptyl group.

In one or a plurality of embodiments, in Formula (III), examples of heteroaryl (including heteroaryl in a heteroaryl methyl group) include: a five- or six-membered monocyclic group including one or two nitrogen atom(s); a five- or six-membered monocyclic group including one or two nitrogen atom(s) and either one oxygen atom or one sulfur atom; a five-membered monocyclic group including one oxygen atom or one sulfur atom; and a bicyclic group including one to four nitrogen atom(s) and formed by condensation of a six-membered ring and a five- or six-membered ring. In one or a plurality of embodiments, other examples include: 2-pyridyl, 3-pyridyl, 4-pyridyl, 2-thienyl, 3-thienyl, 3-oxadiazolyl, 2-imidazolyl, 2-thiazolyl, 3-isothiazolyl, 2-oxadiazolyl, 3-isoxadiazolyl, 2-furyl, 3-furyl, 3-pyrrolyl, 2-quinolyl, 8-quinolyl, 2-quinazolinyl, and 8-purinyl. Examples of the aryl group include an aryl group having a carbon number of not more than 10, such as a phenyl group and a naphthyl group.

In Formulae (I) and (III), one or a plurality of identical or different substituent(s) for a phenyl group, a monocyclic heteroaromatic group and cyclic aliphatic group, and an aryl group and a heteroaryl group (including heteroaryl in a heteroaryl methyl group) may be included. In one or a plurality of embodiments, the examples include a halogen atom, a cyano group, a trifluoromethyl group, a nitro group, a hydroxyl group, a methylenedioxy group, a lower alkyl group, a lower alkoxy group, a benzyloxy group, a lower alkanoyloxy group, an amino group, a mono-lower alkylamino group, a di-lower alkylamino group, a carbamoyl group, a lower alkylamino carbonyl group, a di-lower alkylamino carbonyl group, a carboxyl group, a lower alkoxycarbonyl group, a lower alkylthio group, a lower alkyl sulfinyl group, a lower alkylsulfonyl group, a lower alkanoylamino group, or a lower alkylsulfonamide group. In one or a plurality of embodiments, examples of the halogen atom include an atom of fluorine, chlorine, bromine or iodine. In one or a plurality of embodiments, an example of the lower alkyl is “C₁₋₆ alkyl group” as defined above.

In the present disclosure, a “pharmaceutically acceptable salt” includes a salt that is acceptable from a pharmacologic and/or medical viewpoint, and the examples include: inorganic acid salt, organic acid salt, inorganic basic salt, organic basic salt, and acidic or basic amino acid salt.

Preferred examples of the inorganic acid salt include hydrochloride, hydrobromate, sulfate, nitrate and phosphate. Preferred examples of the organic acid salt include: acetate, succinate, fumarate, maleate, tartarate, citrate, lactate, stearate, benzoate, methane sulfonate, and p-toluene sulfonate.

Preferred examples of the inorganic basic salt include: alkali metal salts such as sodium salt and potassium salt; alkaline earth metal salts such as calcium salt and magnesium salt; aluminum salt, and ammonium salt. Preferred examples of the organic basic salt include: diethylamine salt, diethanolamine salt, meglumine salt, and N,N′-dibenzylethylenediamine salt.

Preferred examples of the acidic amino acid salt include: aspartate and glutamate. Preferred examples of the basic amino acid salt include: arginine salt, lysine salt, and ornithine salt.

In the present disclosure, a “salt of compound” can embrace a hydrate that may be formed from a compound that is exposed to the air so as to absorb moisture.

Further in the present disclosure, a “salt of compound” can embrace a solvate that can be formed from a compound absorbing a kind of solvent.

In the above General formula (I), R¹ is either a hydrogen atom or a C₁₋₆ alkyl group. In one or a plurality of embodiments, R¹ is a hydrogen atom, a methyl group or an ethyl group. In the above General formula (I), R² is selected from the group consisting of —R³, —C≡C—R³, —CH═CH—R³, and —O—(CH₂)ₙ—R³, and n is 1 to 6. In one or a plurality of embodiments, R² is —R³, or —C≡C—R³. In the above General formula (I), R³ is selected from the group consisting of a hydrogen atom, a hydroxyl group, a C₁₋₈ alkyl group or —Si(R⁵)₃, and a substituted or an unsubstituted phenyl group, monocyclic heteroaromatic group and cyclic aliphatic group; or R¹ and R² are bound to each other to form a ring, —R¹-R²— is selected from the group consisting of —(CH₂)ₘ—CH₂— —CH═CH—, —(CH₂)ₘ—O—, and those substituted with halogen atoms, and m is 1 to 6. In one or a plurality of embodiments, R³ is selected from the group consisting of —Si(R⁵)₃ and substituted or unsubstituted phenyl group or cyclic aliphatic group. In one or a plurality of embodiments, R³ is selected from the group consisting of —Si(R⁵)₃, an adamantyl group, and, a phenyl group that may be substituted with one or a plurality of methyl group(s), trifluoromethyl group(s) or hydroxyl group(s), or a cyclohexyl group. In the above General formula (I), R⁴ is either a hydrogen atom or a C₁₋₆ alkyl group. In one or a plurality of embodiments, R⁴ is a hydrogen atom. In one or a plurality of embodiments, in the above General formula (I), R¹ is either a hydrogen atom or a methyl group, R² is —R³ or —C≡C—R³, R³ is selected from the group consisting of —Si(CH₃)₃, an adamantyl group, and, a phenyl group that may be substituted with one or a plurality of methyl group(s) or hydroxyl group(s), or a cyclohexyl group. R⁴ is either a hydrogen atom or a C₁₋₆ alkyl group. R⁵ is either a hydrogen atom or a C₁₋₆ alkyl group, and the three R⁵ in —Si(R⁵)₃ may be different from each other.

In one or a plurality of embodiments, the compound represented by the above General formula (I) or the pharmaceutically acceptable salt thereof is a compound expressed by:

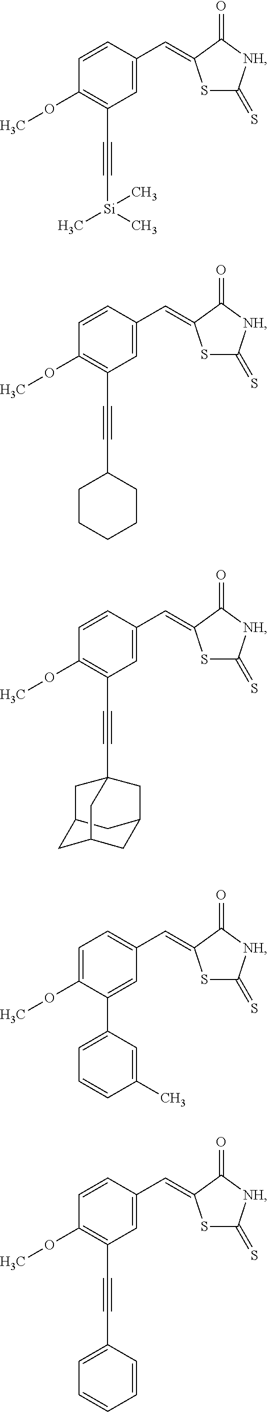

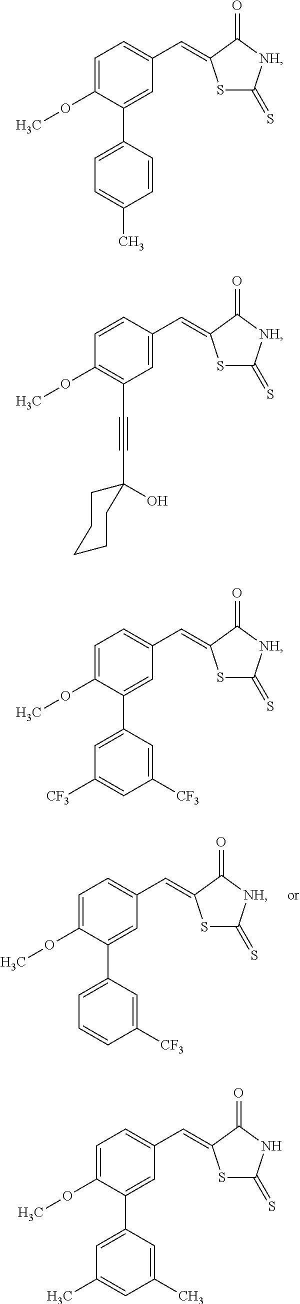

or the pharmaceutically acceptable salt thereof.

In one or a plurality of embodiments, the compound represented by the above General formula (I) or the pharmaceutically acceptable salt thereof is a compound expressed by:

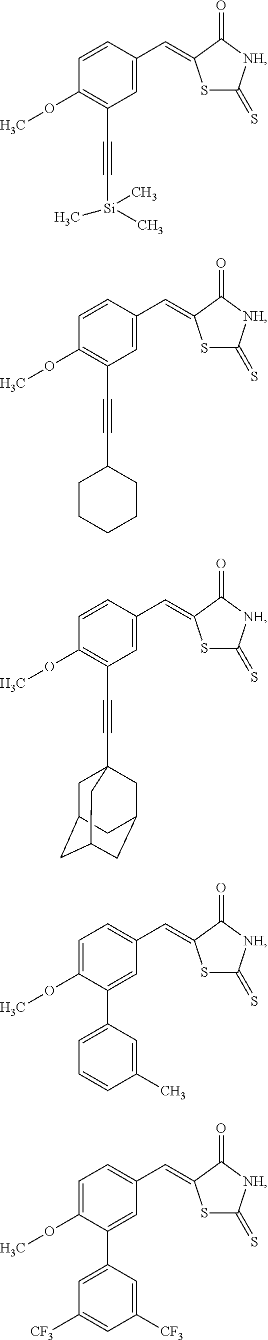

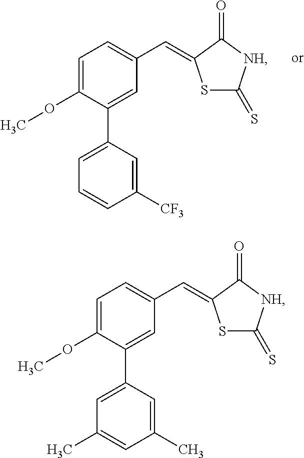

or the pharmaceutically acceptable salt thereof.

In one or a plurality of embodiments, in the General formula (III), R²¹ is either a hydrogen atom or a C₁₋₃ alkyl group. In one or a plurality of embodiments, R²² is either —R²⁶ or —C≡C—R²⁶. In one or a plurality of embodiments, R²⁶ is —Si(R²⁷)₃, or selected from the group consisting of a substituted or an unsubstituted phenyl group, monocyclic heteroaromatic group and cyclic aliphatic group. In one or a plurality of embodiments, R²⁷ is a C₁₋₃ alkyl group. In one or a plurality of embodiments, R²³ is either a hydrogen atom or a C₁₋₆ alkyl group. In one or a plurality of embodiments, R²⁴ and R²⁵ are hydrogen atoms or C₁₋₃ alkyl groups.

Further in one or a plurality of embodiments, the compound represented by General formula (III) does not include Harmine. Further, in one or a plurality of embodiments, it is not a combination to allow R²¹, R²², R²³, R²⁴, and R²⁵ in General formula (III) to make Harmine (i.e., a combination where R²¹ is a methyl group, R²² and R²³ are hydrogen atoms, R²⁴ is a methyl group, and R²⁵ is a hydrogen atom).

In one or a plurality of embodiments, the compound represented by the above General formula (III) or the pharmaceutically acceptable salt thereof is represented by:

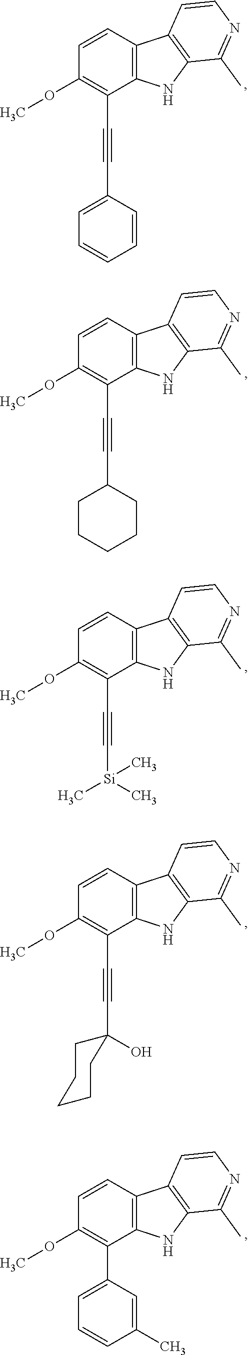

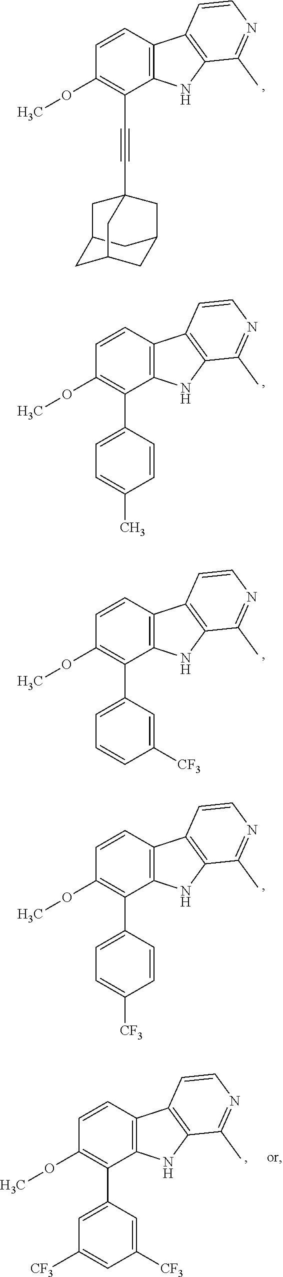

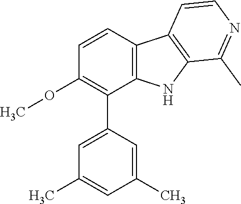

or a pharmaceutically acceptable salt thereof.

In one or a plurality of embodiments, the compound represented by the above General formula (III) or the pharmaceutically acceptable salt thereof is represented by:

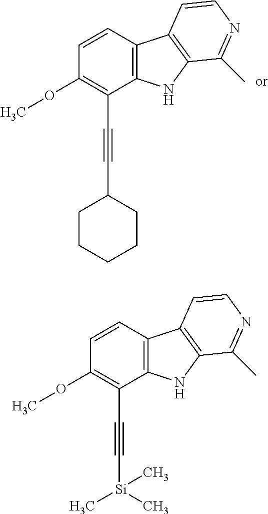

or a pharmaceutically acceptable salt thereof.

In one or a plurality of embodiments, the compounds represented by the above General formulae (I) and (III) or the pharmaceutically acceptable salts thereof are capable of inducing instability in an in vivo or intracellular DYRK1A protein or reducing the amount of an in vivo or intracellular DYRK1A protein.

In one or a plurality of embodiments, “intracellular” in the present disclosure may indicate the interior of an in vivo, in vitro or ex vivo cell.

Therefore, the present disclosure relates to a composition for inducing instability in an in vivo or intracellular DYRK1A protein or for reducing the amount of an in vivo or intracellular DYRK1A protein, the composition includes the compound represented by the above General formula (I) or (III) or the pharmaceutically acceptable salt thereof. Further the present disclosure relates to a compound represented by the above General formula (I) or (III) or a pharmaceutically acceptable salt thereof for inducing instability in an in vivo or intracellular DYRK1A protein, or for reducing the amount of an in vivo or intracellular DYRK1A protein. Furthermore, the present disclosure relates to use of a compound represented by the above General formula (I) or (III) or a pharmaceutically acceptable salt thereof for producing a composition for inducing instability in an in vivo or intracellular DYRK1A protein or for reducing the amount of an in vivo or intracellular DYRK1A protein.

### [Method Relating to Induction of Instability in DYRK1A]

In one or a plurality of embodiments, the present disclosure relates to a method for inducing instability in an in vivo or intracellular DYRK1A protein or for reducing the amount of an in vivo or intracellular DYRK1A protein. The method includes administration of the compound represented by the above General formula (I) or (III) or the pharmaceutically acceptable salt thereof to a living body or a cell. In one or a plurality of embodiments, the living body or the cell is a living body or a cell that expresses the DYRK1A protein.

[Prevention, Improvement, Suppression of Progression and/or Treatment of Alzheimer's Disease]

It is indicated that phosphoenzyme DYRK1A is over-produced in Down's syndrome, and this overproduction is considered as causing Alzheimer's disease that develops with high probability in Down's syndrome. Therefore, in one or a plurality of embodiments, the present disclosure relates to prevention, improvement, suppression of progression and/or treatment of Alzheimer's disease, by use of a compound, a pharmaceutically acceptable salt thereof, or a composition including the same, for inducing instability in an in vivo or intracellular DYRK1A protein or for reducing the amount of an in vivo or intracellular DYRK1A protein. In one or a plurality of embodiments, the above-mentioned prevention, improvement, suppression of progression and/or treatment of Alzheimer's disease is prevention, improvement, suppression of progression and/or treatment of Alzheimer's disease that can develop in Down's syndrome.

In one or a plurality of embodiments, the present disclosure relates to a pharmaceutical composition for prevention, improvement, suppression of progression and/or treatment of Alzheimer's disease, which contains the compound represented by the above General formula (I) or (III) or the pharmaceutically acceptable salt thereof as the active ingredient (hereinafter, this is stated also as “pharmaceutical composition D according to the present disclosure”). Further, in one or a plurality of embodiments, the present disclosure relates to the compound represented by the above General formula (I) or (III) or the pharmaceutically acceptable salt thereof for prevention, improvement, suppression of progression and/or treatment of Alzheimer's disease. Furthermore, in one or a plurality of embodiments, the present disclosure relates to use of the compound represented by the above General formula (I) or (III) or the pharmaceutically acceptable salt thereof for producing a pharmaceutical composition for prevention, improvement, suppression of progression and/or treatment of Alzheimer's disease.

In one or a plurality of embodiments, a “pharmaceutical composition” in the present disclosure can be prepared in dosage forms suitable for administration forms by application of known pharmaceutical techniques. Though there is no particular limitation, an example of the dosage form is oral administration in the form of: tablets, capsules, granules, powder, pills, lozenges, syrup, and liquid medicines. Another example is parenteral administration in the form of injections, liquid medicines, aerosol, suppository, patches, poultices, lotions, liniments, ointments, instillations and the like. These medicines can be produced in known methods by use of additives such as vehicles, lubricants, binders, disintegrators, stabilizers, corrigents, and diluents, though the present disclosure is not limited thereto.

Examples of the vehicle include: starches such as starch, potato starch and corn starch; lactose, crystalline cellulose, and calcium hydrogen phosphate, though the present disclosure is not limited thereto. Examples of the coating agent include; ethyl cellulose, hydroxypropylcellulose, hydroxypropylmethylcellulose, shellac, talc, carnauba wax, and paraffin, though the present disclosure is not limited thereto. Examples of the binder include: polyvinyl pyrrolidone, macrogol and the compound similar to those for the vehicles, though the present disclosure is not limited thereto. Examples of the disintegrator include: the compounds similar to those for the above-mentioned vehicles; and chemically modified starches and celluloses such as croscarmellose sodium, carboxymethyl starch sodium and crosslinked polyvinyl pyrrolidone, though the present disclosure is not limited thereto. Examples of the stabilizer include: parahydroxybenzoate esters such as methylparaben and propylparaben; alcohols such as chlorobutanol, benzyl alcohol and phenylethyl alcohol; benzalkonium chloride; phenols such as phenol and cresol; Thimerosal; dehydroacetic acid; and sorbic acid, though the present disclosure is not limited thereto. Examples of the corrigents include sweeteners, acidifiers, aroma chemicals and the like, which are commonly used, though the present disclosure is not limited thereto.

In preparation of a liquid medicine, ethanol, phenol, chlorocresol, purified water, distilled water and the like can be used as the solvent, though the present disclosure is not limited thereto. If necessary, a surfactant, an emulsifier or the like can be used as well. Examples of the surfactant or the emulsifier include Polysorbate 80, Polyoxyl stearate 40, and Lauromacrogol, though the present disclosure is not limited thereto.

The method of using the pharmaceutical composition D according to the present disclosure can vary depending on symptoms, ages, administration methods and the like. In the method of use, it is possible to administer the compound intermittently or continuously in an oral, endermic, submucous, subcutaneous, intramuscular, intravascular, intracerebral, or intraperitoneal manner, though the present disclosure is not limited thereto, so that the in vivo concentration of the compound as the active ingredient represented by the above General formula (I) or (III) will be in the range of 100 nM to 1 mM. In an unlimited embodiment, in a case of oral administration, for example, a dose of not less than 0.01 mg (preferably, 0.1 mg) and not more than 2000 mg (preferably 500 mg, and more preferably 100 mg) per day in terms of the compound represented by the above General formula (I) or (III) is administered to a subject (in a case of human being, adult) at a time or several times in accordance with the symptoms. In an unlimited embodiment, in a case of intravenous administration, a dose of not less than 0.001 mg (preferably, 0.01 mg) and not more than 500 mg (preferably 50 mg) per day is administered to a subject (in a case of human being, adult) at a time or several times in accordance with the symptoms.

In one or a plurality of embodiments, the present disclosure relates to a method for prevention, improvement, suppression of progression and/or treatment of Alzheimer's disease, including administration of the compound represented by the above General formula (I) or (III) or the pharmaceutically acceptable salt thereof to a subject. In one or a plurality of embodiments, the administration of the compound represented by the above General formula (I) or (III) or the pharmaceutically acceptable salt thereof can correspond to the above-mentioned method of use of the pharmaceutical composition D. The subjects may include human beings and animals other than human beings. Examples of the animals include animals that express the DYRK1A protein.

Namely, the present disclosure can relate to one or a plurality of the following embodiments.

[D1] A compound represented by General formula (I) below or a pharmaceutically acceptable salt thereof.

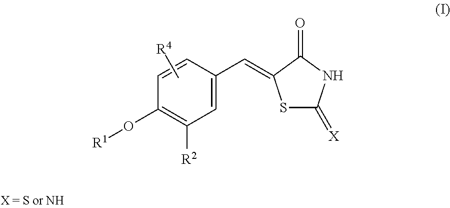

[In Formula (I), R¹ is either a hydrogen atom or a C₁₋₆ alkyl group; R² is selected from the group consisting of —R³, —C≡C—R³, —CH═CH—R³ and —O—(CH₂)ₙ—R³, where n is 1 to 6; R³ is selected from the group consisting of a hydrogen atom, a hydroxyl group, a C₁₋₈ alkyl group, —Si(R⁵)₃, and, a substituted or unsubstituted phenyl group, a monocyclic heteroaromatic group and a cyclic aliphatic group; or R¹ and R² are bonded to each other to form a ring, where —R¹-R²— is selected from the group consisting of —(CH₂)ₘ—CH₂—, —CH═CH—, —(CH₂)ₘ—O— and those substituted with halogen atoms, where m is 1 to 6; R⁴ is either a hydrogen atom or a C₁₋₆ alkyl group; and R⁵ is either a hydrogen atom or a C₁₋₆ alkyl group, where the three R⁵ in —Si(R⁵)₃ may be different from each other.]

[D2] A compound represented by:

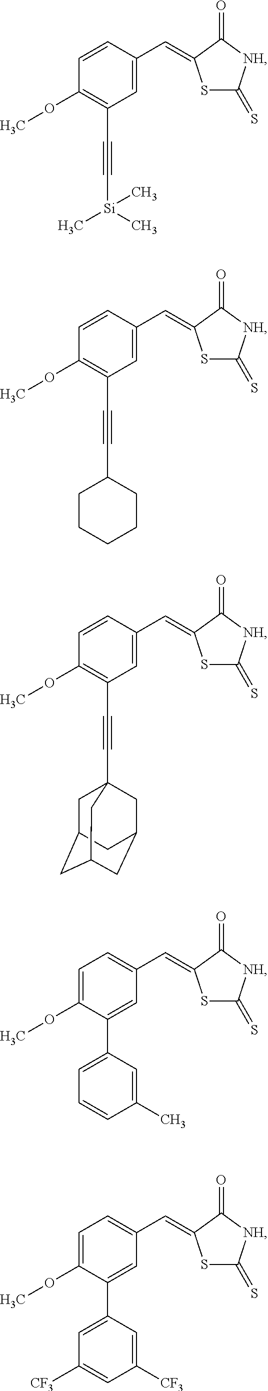 [SMILES: COc1ccc(/C=C2\SC(=S)NC2=O)cc1C#C[Si](C)(C)C ; COc1ccc(/C=C2\SC(=S)NC2=O)cc1C#CC1CCCCC1 ; COc1ccc(/C=C2\SC(=S)NC2=O)cc1C#CC12CC3CC(CC(C3)C1)C2 ; COc1ccc(/C=C2\SC(=S)NC2=O)cc1-c1cccc(C)c1 ; COc1ccc(/C=C2\SC(=S)NC2=O)cc1-c1cc(C(F)(F)F)cc(C(F)(F)F)c1]

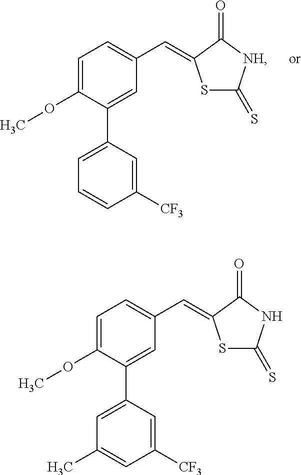 [SMILES: COc1ccc(/C=C2\SC(=S)NC2=O)cc1-c1cccc(C(F)(F)F)c1 ; "or" ; COc1ccc(/C=C2\SC(=S)NC2=O)cc1-c1cc(C)cc(C(F)(F)F)c1]

or a pharmaceutically acceptable salt thereof.
[D3] A composition for inducing instability in an in vivo or intracellular DYRK1A protein or for reducing the amount of an in vivo or intracellular DYRK1A protein, including the compound as recited in [D1] or [D2] or the pharmaceutically acceptable salt thereof.
[D4] The compound as recited in [D1] or [D2] or the pharmaceutically acceptable salt thereof for inducing instability in an in vivo or intracellular DYRK1A protein or for reducing the amount of an in vivo or intracellular DYRK1A protein.
[D5] Use of the compound as recited in [D1] or [D2] or the pharmaceutically acceptable salt thereof for producing a composition for inducing instability in an in vivo or intracellular DYRK1A protein or for reducing the amount of an in vivo or intracellular DYRK1A protein.
[D6] A method for inducing instability in an in vivo or intracellular DYRK1A protein or for reducing the amount of an in vivo or intracellular DYRK1A protein, the method including administration of a compound represented by General formula (I) below or a pharmaceutically acceptable salt thereof to a living body or a cell.

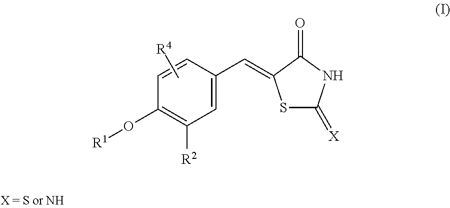

[In Formula (I), R¹ is either a hydrogen atom or a C₁₋₆ alkyl group; R² is selected from the group consisting of —R³, —C≡C—R³, —CH═CH—R³ and —O—(CH₂)ₙ—R³, where n is 1 to 6;

R³ is selected from the group consisting of a hydrogen atom, a hydroxyl group, a C₁₋₈ alkyl group, —Si(R⁵)₃, and, a substituted or unsubstituted phenyl group, a monocyclic heteroaromatic group and a cyclic aliphatic group; or R¹ and R² are bonded to each other to form a ring, where —R¹-R²— is selected from the group consisting of —(CH₂)ₘ—CH₂—, —CH═CH—, —(CH₂)ₘ—O— and those substituted with halogen atoms, where m is 1 to 6; R⁴ is either a hydrogen atom or a C₁₋₆ alkyl group; and R⁵ is either a hydrogen atom or a C₁₋₆ alkyl group, where the three R⁵ in —Si(R⁵)₃ may be different from each other.]

[D7] A method for inducing instability in an in vivo or intracellular DYRK1A protein or for reducing the amount of an in vivo or intracellular DYRK1A protein, the method including administration of a compound represented by:

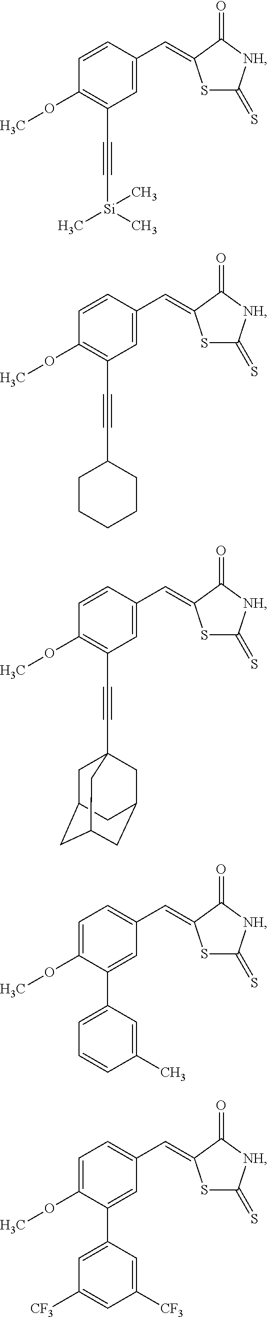 [SMILES: COc1ccc(/C=C2\SC(=S)NC2=O)cc1C#C[Si](C)(C)C ; COc1ccc(/C=C2\SC(=S)NC2=O)cc1C#CC1CCCCC1 ; COc1ccc(/C=C2\SC(=S)NC2=O)cc1C#CC12CC3CC(CC(C3)C1)C2 ; COc1ccc(/C=C2\SC(=S)NC2=O)cc1-c1cccc(C)c1 ; COc1ccc(/C=C2\SC(=S)NC2=O)cc1-c1cc(C(F)(F)F)cc(C(F)(F)F)c1]

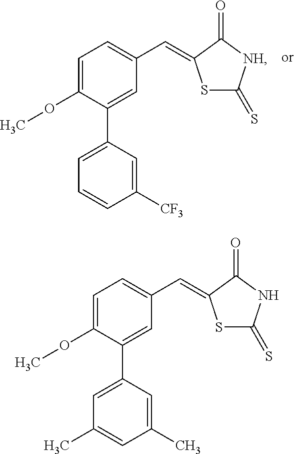 [SMILES: COc1ccc(/C=C2\SC(=S)NC2=O)cc1-c1cccc(C(F)(F)F)c1 ; "or" ; COc1ccc(/C=C2\SC(=S)NC2=O)cc1-c1cc(C)cc(C)c1]

or a pharmaceutically acceptable salt thereof to a living body or a cell.
[D8] A pharmaceutical composition for prevention, improvement, suppression of progression and/or treatment of Alzheimer's disease, containing the compound as recited in [D1] or [D2] or the pharmaceutically acceptable salt thereof as an active ingredient.
[D9] The compound as recited in [D1] or [D2] or the pharmaceutically acceptable salt thereof for prevention, improvement, suppression of progression and/or treatment of Alzheimer's disease.
[D10] Use of the compound as recited in [D1] or [D2] or the pharmaceutically acceptable salt thereof for production of a pharmaceutical composition for prevention, improvement, suppression of progression and/or treatment of Alzheimer's disease.
[D11] A method for prevention, improvement, suppression of progression and/or treatment of Alzheimer's disease, including administration of a compound represented by General formula (I) below or a pharmaceutically acceptable salt thereof to a subject:

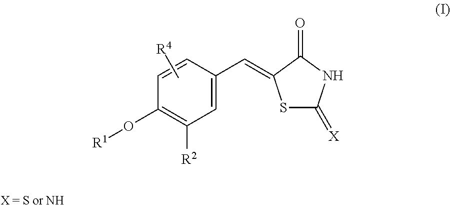

[In Formula (I), R¹ is either a hydrogen atom or a C₁₋₆ alkyl group; R² is selected from the group consisting of —R³, —C≡C—R³, —CH═CH—R³ and —O—(CH₂)ₙ—R³, where n is 1 to 6; R³ is selected from the group consisting of a hydrogen atom, a hydroxyl group, a C₁₋₈ alkyl group, —Si(R⁵)₃, and, a substituted or unsubstituted phenyl group, a monocyclic heteroaromatic group and a cyclic aliphatic group; or R¹ and R² are bonded to each other to form a ring, where —R¹-R²— is selected from the group consisting of —(CH₂)ₘ—CH₂—, —CH═CH—, —(CH₂)ₘ—O— and those substituted with halogen atoms, where m is 1 to 6; R⁴ is either a hydrogen atom or a C₁₋₆ alkyl group; and R⁵ is either a hydrogen atom or a C₁₋₆ alkyl group, where the three R⁵ in —Si(R⁵)₃ may be different from each other.]

[D12] A method for prevention, improvement, suppression of progression and/or treatment of Alzheimer's disease, including administration of a compound represented by:

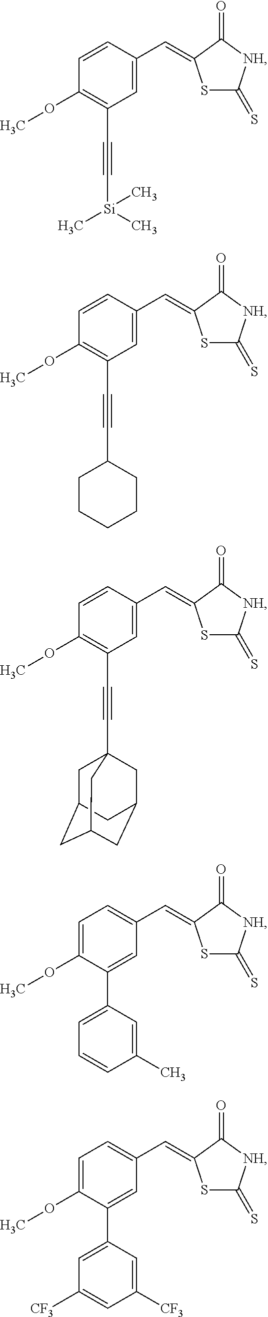 [SMILES: COc1ccc(/C=C2\SC(=S)NC2=O)cc1C#C[Si](C)(C)C ; COc1ccc(/C=C2\SC(=S)NC2=O)cc1C#CC1CCCCC1 ; COc1ccc(/C=C2\SC(=S)NC2=O)cc1C#CC12CC3CC(CC(C3)C1)C2 ; COc1ccc(/C=C2\SC(=S)NC2=O)cc1-c1cccc(C)c1 ; COc1ccc(/C=C2\SC(=S)NC2=O)cc1-c1cc(C(F)(F)F)cc(C(F)(F)F)c1]

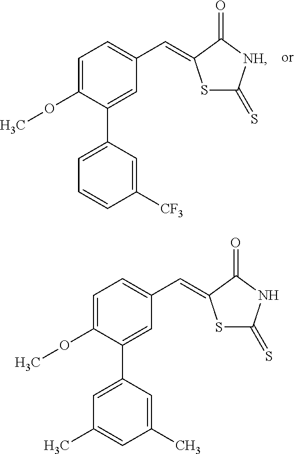 [SMILES: COc1ccc(/C=C2\SC(=S)NC2=O)cc1-c1cccc(C(F)(F)F)c1 ; "or" ; COc1ccc(/C=C2\SC(=S)NC2=O)cc1-c1cc(C)cc(C)c1]

or a pharmaceutically acceptable salt thereof to a subject.
[D13] The pharmaceutical composition, the salt, the use or the method according to any one of [D8] to [D12], wherein the Alzheimer's disease is Alzheimer's disease that can develop in Down's syndrome.

Further, the present disclosure can relate to the following one or a plurality of embodiments.

[D′1] A compound represented by General formula (III) below or a pharmaceutically acceptable salt thereof.

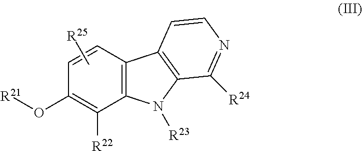

[In Formula (III), R²¹ and R²³ each independently is a hydrogen atom, a C₁₋₆ linear or branched or cyclic alkyl group, a benzyl or heteroaryl methyl group, a substituted or unsubstituted aryl group, or a substituted or unsubstituted heteroaryl group; R²² is selected from the group consisting of —R²⁶, —C≡C—R²⁶, —CH═CH—R²⁶ and —O—(CH₂)n-R²⁶, where n is 1 to 6; R²⁶ is selected from the group consisting of a hydrogen atom, a hydroxyl group, a C₁₋₈ alkyl group, —Si(R²⁷)₃, and, a substituted or unsubstituted phenyl group, a monocyclic heteroaromatic group and cyclic aliphatic group; or, R²¹ and R²² are bonded to each other to form a ring, —R²¹-R²²— is selected from the group consisting of —(CH₂)m-CH₂—, —CH═CH—, —(CH₂)m-O— and those substituted with a halogen atom, where m is 1 to 6; R²⁷ is a hydrogen atom, a C₁₋₆ alkyl group, a trihalomethyl group, or a hydroxyl group, and three R²⁷ in —Si(R²⁷)₃ may be different from each other; and R²⁴, R²⁵ are either hydrogen atoms or C₁ alkyl groups.]

[D′2] A compound represented by:

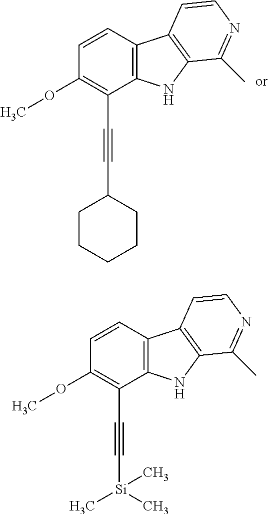

or a pharmaceutically acceptable salt thereof.
[D′3] A composition for inducing instability in an in vivo or intracellular DYRK1A protein or for reducing the amount of an in vivo or intracellular DYRK1A protein, including the compound as recited in [D′1] or [D′2] or the pharmaceutically acceptable salt thereof.
[D′4] The compound as recited in [D′1] or [D′2] or the pharmaceutically acceptable salt thereof for inducing instability in an in vivo or intracellular DYRK1A protein or for reducing the amount of an in vivo or intracellular DYRK1A protein.
[D′5] Use of the compound as recited in [D′1] or [D′2] or the pharmaceutically acceptable salt thereof for producing a composition for inducing instability in an in vivo or intracellular DYRK1A protein or for reducing the amount of an in vivo or intracellular DYRK1A protein.
[D′6] A method for inducing instability in an in vivo or intracellular DYRK1A protein or for reducing the amount of an in vivo or intracellular DYRK1A protein, the method including administration of a compound represented by General formula (III) below or a pharmaceutically acceptable salt thereof to a living body or a cell.

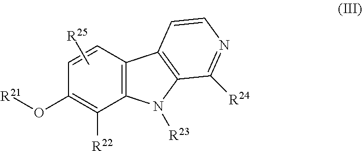

[In Formula (III), R²¹ and R²³ each independently is a hydrogen atom, a C₁₋₆ linear or branched or cyclic alkyl group, a benzyl or heteroaryl methyl group, a substituted or unsubstituted aryl group, or a substituted or unsubstituted heteroaryl group; R²² is selected from the group consisting of —R²⁶, —C≡C—R²⁶, —CH═CH—R²⁶ and —O—(CH₂)n-R²⁶, where n is 1 to 6; R²⁶ is selected from the group consisting of a hydrogen atom, a hydroxyl group, a C₁₋₈ alkyl group, —Si(R²⁷)₃, and, a substituted or unsubstituted phenyl group, a monocyclic heteroaromatic group and cyclic aliphatic group; or, R²¹ and R²² are bonded to each other to form a ring, —R²¹-R²²— is selected from the group consisting of —(CH₂)m-CH₂—, —CH═CH—, —(CH₂)m-O— and those substituted with a halogen atom, where m is 1 to 6; R²⁷ is a hydrogen atom, a C₁₋₆ alkyl group, a trihalomethyl group, or a hydroxyl group, and three R²⁷ in —Si(R²⁷)₃ may be different from each other; and R²⁴, R²⁵ are either hydrogen atoms or C₁₋₆ alkyl groups.]
[D′7] A method for inducing instability in an in vivo or intracellular DYRK1A protein or for reducing the amount of an in vivo or intracellular DYRK1A protein, the method including administration of a compound represented by:

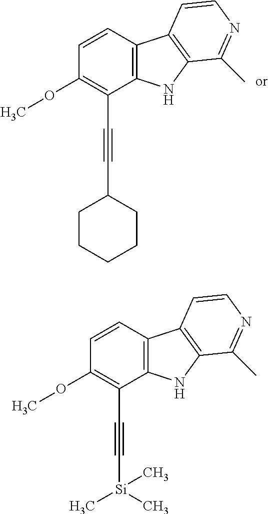

or a pharmaceutically acceptable salt thereof to a living body or a cell.
[D′8] A pharmaceutical composition for prevention, improvement, suppression of progression and/or treatment of Alzheimer's disease, containing the compound as recited in [D′1] or [D′2] or the pharmaceutically acceptable salt thereof as an active ingredient.
[D′9] The compound or the pharmaceutically acceptable salt thereof as recited in
[D′1] or [D′2] for prevention, improvement, suppression of progression and/or treatment of Alzheimer's disease.
[D′10] Use of the compound as recited in [D′1] or [D′2] or the pharmaceutically acceptable salt thereof for production of a pharmaceutical composition for prevention, improvement, suppression of progression and/or treatment of Alzheimer's disease.
[D′11] A method for prevention, improvement, suppression of progression and/or treatment of Alzheimer's disease, including administration of a compound represented by General formula (III) below or a pharmaceutically acceptable salt thereof to a subject:

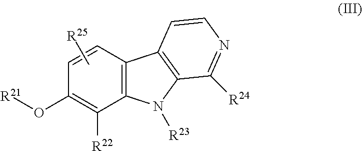

[In Formula (III), R²¹ and R²³ each independently is a hydrogen atom, a C₁₋₆ linear or branched or cyclic alkyl group, a benzyl or heteroaryl methyl group, a substituted or unsubstituted aryl group, or a substituted or unsubstituted heteroaryl group; R²² is selected from the group consisting of —R²⁶, —C≡C—R²⁶, —CH═CH—R²⁶ and —O—(CH₂)n-R²⁶, where n is 1 to 6; R²⁶ is selected from the group consisting of a hydrogen atom, a hydroxyl group, a C₁₋₈ alkyl group, —Si(R²⁷)₃, and, a substituted or unsubstituted phenyl group, a monocyclic heteroaromatic group and cyclic aliphatic group; or, R²¹ and R²² are bonded to each other to form a ring, —R²¹-R²²— is selected from the group consisting of —(CH₂)m-CH₂—, —CH═CH—, —(CH₂)m-O— and those substituted with a halogen atom, where m is 1 to 6; R²⁷ is a hydrogen atom, a C₁₋₆ alkyl group, a trihalomethyl group, or a hydroxyl group, and three R²⁷ in —Si(R²⁷)₃ may be different from each other; and R²⁴, R²⁵ are either hydrogen atoms or C₁₋₆ alkyl groups.]
[D′12] A method for prevention, improvement, suppression of progression and/or treatment of Alzheimer's disease, including administration of a compound represented by:

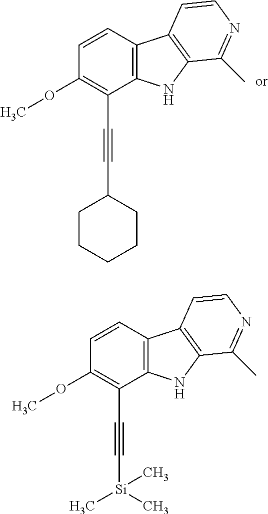

or a pharmaceutically acceptable salt thereof to a subject.
[D′13] The pharmaceutical composition, the salt, the use or the method according to any of [D′8] to [D′12], wherein the Alzheimer's disease is Alzheimer's disease that can develop in Down's syndrome.

### [Substance Relating to Induction of Instability in TAU]

In one or a plurality of embodiments, the present disclosure relates to a compound represented by the following General formula (II) or a pharmaceutically acceptable salt thereof:

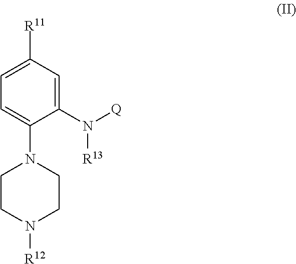

[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

In one or a plurality of embodiments, the compound represented by the General formula (II) above or the pharmaceutically acceptable salt thereof is a compound represented by:

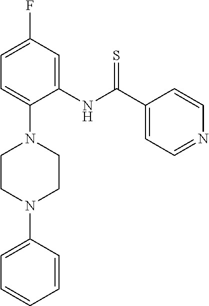

or the pharmaceutically acceptable salt thereof.

In one or a plurality of embodiments, the compound represented by the above General formula (II) or the pharmaceutically acceptable salt thereof is capable of reducing instability in an in vivo or intracellular TAU protein or reducing the amount of an in vivo or intracellular TAU protein.

Therefore, the present disclosure relates to a composition for inducing instability in an in vivo or intracellular TAU protein or for reducing the amount of an in vivo or intracellular TAU protein, including the compound represented by the above General formula (II) or the pharmaceutically acceptable salt thereof. Further, the present disclosure relates to a compound represented by the above General formula (II) or a pharmaceutically acceptable salt thereof for inducing instability in an in vivo or intracellular TAU protein or for reducing the amount of an in vivo or intracellular TAU protein. Furthermore, the present disclosure relates to use of a compound represented by the above General formula (II) or a pharmaceutically acceptable salt thereof for producing a composition for inducing instability in an in vivo or intracellular TAU protein or for reducing the amount of an in vivo or intracellular TAU protein.

### [Method Relating to Induction of Instability in TAU]

In one or a plurality of embodiments, the present disclosure relates to a method for inducing instability in an in vivo or intracellular TAU protein or for reducing the amount of an in vivo or intracellular TAU protein. The method includes administration of the compound represented by the above General formula (II) or the pharmaceutically acceptable salt thereof to a living body or a cell. In one or a plurality of embodiments, the living body or the cell is a living body or a cell that expresses TAU protein.

[Prevention, Improvement, Suppression of Progression and/or Treatment of Alzheimer's Disease or Tauopathies]

It is considered that microtubule connected protein TAU is insolubilized and accumulated as a result of over-phosphorylation, and that the accumulation of over-phosphorylated TAU is the critical cause of neurodegeneration disease. It has been shown from former and conventional analyses using gene-deleted mice that development of Alzheimer's disease can be suppressed by deleting the TAU gene. This result implies that development of Alzheimer's disease can be suppressed by reducing TAU gene product (TAU protein). Further, accumulation of TAU protein is regarded as the cause of a dementia, i.e., Tauopathies. Therefore, in one or a plurality of embodiments, the present disclosure relates to a method for prevention, improvement, suppression of progression and/or treatment of Alzheimer's disease or Tauopathies, using a compound for inducing instability in an in vivo or intracellular TAU protein or for reducing the amount of an in vivo or intracellular TAU protein, or a pharmaceutically acceptable salt thereof, or a composition including the same.

In one or a plurality of embodiments, the present disclosure relates to a pharmaceutical composition for prevention, improvement, suppression of progression and/or treatment of Alzheimer's disease or Tauopathies, which contains the compound represented by the above General formula (II) or the pharmaceutically acceptable salt thereof as the active ingredient (hereinafter, this is stated also as “pharmaceutical composition T according to the present disclosure”). Further, in one or a plurality of embodiments, the present disclosure relates to a compound represented by the above General formula (II) or the pharmaceutically acceptable salt thereof for prevention, improvement, suppression of progression and/or treatment of Alzheimer's disease or Tauopathies. Furthermore, in one or a plurality of embodiments, the present disclosure relates to use of the compound represented by the above General formula (II) or the pharmaceutically acceptable salt thereof for prevention, improvement, suppression of progression and/or treatment of Alzheimer's disease or Tauopathies.

The method of using the pharmaceutical composition T according to the present disclosure can vary depending on symptoms, ages, administration methods and the like. In the method of use, it is possible to administer the compound intermittently or continuously in an oral, endermic, submucous, subcutaneous, intramuscular, intravascular, intracerebral, or intraperitoneal manner, though the present disclosure is not limited thereto, so that the intracorporeal concentration of the compound represented by the above General formula (II) will be in the range of 100 nM to 1 mM. In an unlimited embodiment, in a case of oral administration, for example, a dose of not less than 0.01 mg (preferably 0.1 mg) and not more than 2000 mg (preferably 500 mg, and more preferably 100 mg) per day in terms of the compound represented by the above General formula (II) is administered to a subject (in a case of human being, adult) at a time or several times in accordance with the symptoms. In an unlimited embodiment, in a case of intravenous administration, a dose of not less than 0.001 mg (preferably 0.01 mg) and not more than 500 mg (preferably 50 mg) per day is administered to a subject (in a case of human being, adult) at a time or several times in accordance with the symptoms.

In one or a plurality of embodiments, the present disclosure relates to a method for prevention, improvement, suppression of progression and/or treatment of Alzheimer's disease or Tauopathies, including administration of the compound represented by the above General formula (II) or the pharmaceutically acceptable salt thereof to a subject. In one or a plurality of embodiments, the administration of the compound represented by the above General formula (II) or the pharmaceutically acceptable salt thereof can correspond to the above-mentioned method of use of the pharmaceutical composition T. The subjects may include human beings and animals other than human beings. Examples of the animals include animals that express TAU protein.

Namely, the present disclosure can relate to one or a plurality of the following embodiments.

[T1] A compound represented by General formula (II) below or a pharmaceutically acceptable salt thereof.

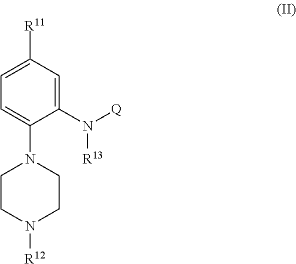

[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group that may be substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group that may be substituted with a C₁₋₆ alkyl group, a C₁₋₆ alkoxy group, a hydroxyl group or a halogen atom, or a monocyclic heteroaromatic group]

[T2] A compound represented by:

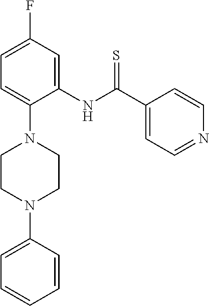 [SMILES: Fc1ccc(N2CCN(c3ccccc3)CC2)c(NC(=S)c2ccncc2)c1]

or a pharmaceutically acceptable salt thereof.
[T3] A composition for inducing instability in an in vivo or intracellular TAU protein or for reducing the amount of an in vivo or intracellular TAU protein, including the compound recited in [T1] or [T2] or the pharmaceutically acceptable salt thereof.
[T4] The compound as recited in [T1] or [T2] or the pharmaceutically acceptable salt thereof for inducing instability in an in vivo or intracellular TAU protein or for reducing the amount of an in vivo or intracellular TAU protein.
[T5] Use of the compound as recited in [T1] or [T2] or the pharmaceutically acceptable salt thereof for producing a composition for inducing instability in an in vivo or intracellular TAU protein or for reducing the amount of an in vivo or intracellular TAU protein.
[T6] A method for inducing instability in an in vivo or intracellular TAU protein or for reducing the amount of an in vivo or intracellular TAU protein, the method including administration of a compound represented by General formula (II) below or a pharmaceutically acceptable salt thereof to a living body or a cell.

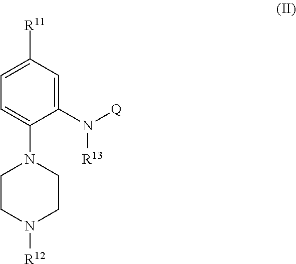

[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group that may be substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group that may be substituted with a C₁₋₆ alkyl group, a C₁₋₆ alkoxy group, a hydroxyl group or a halogen atom, or a monocyclic heteroaromatic group.]

[T7] A method for inducing instability in an in vivo or intracellular TAU protein or for reducing the amount of an in vivo or intracellular TAU protein, the method including administration of a compound represented by:

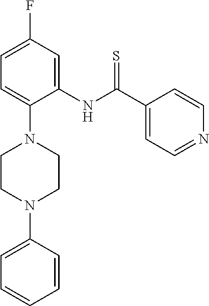 [SMILES: Fc1ccc(N2CCN(c3ccccc3)CC2)c(NC(=S)c2ccncc2)c1]

or a pharmaceutically acceptable salt thereof to a living body or a cell.
[T8] A pharmaceutical composition for prevention, improvement, suppression of progression and/or treatment of Alzheimer's disease or Tauopathies, containing the compound as recited in [T1] or [T2] or the pharmaceutically acceptable salt thereof as an active ingredient.
[T9] The compound as recited in [T1] or [T2] or the pharmaceutically acceptable salt thereof for prevention, improvement, suppression of progression and/or treatment of Alzheimer's disease or Tauopathies.
[T10] Use of the compound as recited in [T1] or [T2] or the pharmaceutically acceptable salt thereof for producing a pharmaceutical composition for prevention, improvement, suppression of progression and/or treatment of Alzheimer's disease or Tauopathies.
[T11] A method for prevention, improvement, suppression of progression and/or treatment of Alzheimer's disease or Tauopathies, including administration of a compound represented by General formula (II) below or a pharmaceutically acceptable salt thereof to a subject:

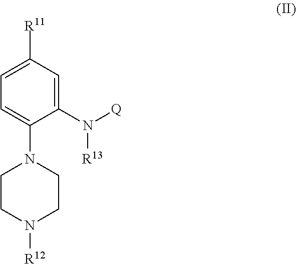

[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group that may be substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group that may be substituted with a C₁₋₆ alkyl group, a C₁₋₆ alkoxy group, a hydroxyl group or a halogen atom, or a monocyclic heteroaromatic group.]

[T12] A method for prevention, improvement, suppression of progression and/or treatment of Alzheimer's disease or Tauopathies, including administration of a compound represented by:

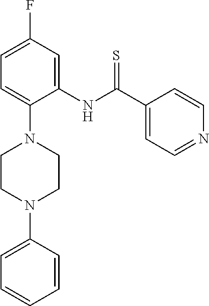 [SMILES: Fc1ccc(N2CCN(c3ccccc3)CC2)c(NC(=S)c2ccncc2)c1]

or a pharmaceutically acceptable salt thereof to a subject.

### [Index of DYRK1A]

As for a patient of Down's syndrome and a mouse model in which overexpression of DYRK1A is recognized, a homocysteine concentration in blood is decreased dramatically. It has been known that this decrease of homocysteine concentration in blood is caused by overexpression of DYRK1A in the liver (Noll et al, PLoS One. 2009). Further, the inventors have found that the homocysteine concentration in blood is raised by administration of DYRK1A inhibitor to a living body (rat). Therefore, in one or a plurality of embodiments, the present disclosure relates to use of homocysteine concentration in blood as an index of in vivo DYRK1A protein activity. Further, in one or a plurality of embodiments, the present disclosure relates to a method for monitoring in vivo DYRK1A protein activity by use of the homocysteine concentration in blood. According to the use of homocysteine concentration in blood of the present disclosure and according to the monitoring method of the present disclosure, for example, it is possible to monitor the in vivo DYRK1A activity inhibition of the same living individual by using the homocysteine concentration in blood as the index. Further, in one or a plurality of embodiments, the use of the homocysteine concentration in blood of the present disclosure and the monitoring method of the present disclosure enable to review the dosage and administration schedule of candidate compounds to be studied regarding DYRK1A activity inhibition while keeping an animal model alive. In addition to that, in one or a plurality of embodiments, use of the homocysteine concentration in blood of the present disclosure and the monitoring method of the present disclosure enable to measure indirectly the in vivo DYRK1A activity of human being.

Overproduction of DYRK1A is considered as causing Alzheimer's disease that develops with high probability in Down's syndrome. Therefore, in one or a plurality of embodiments, the present disclosure relates to a biochemical scoring of Alzheimer's disease or a biochemical assessment of morbidity, including use of the homocysteine concentration in blood of the present disclosure and the monitoring method of the present disclosure. In one or a plurality of embodiments, the Alzheimer's disease is Alzheimer's disease that can develop in Down's syndrome.

In one or a plurality of embodiments, the present disclosure relates to a method of assessing DYRK1A protein activity in an individual, and the method includes monitoring the homocysteine concentration in blood of the individual, and assessing the DYRK1A protein activity in the individual through a comparison of a criterion of assorting that the DYRK1A protein activity is enhanced in a case where the homocysteine concentration in blood is lowered and assorting that the DYRK1A protein activity is suppressed in a case where the homocysteine concentration in blood is raised. In one or a plurality of embodiments, the individual is a living body, and the examples include a human being, a mouse, a rat and any other animal expressing DYRK1A protein.

In one or a plurality of embodiments, the present disclosure relates to a method for assessing an effect of administering a composition including a compound to inhibit DYRK1A activity or a candidate compound, and the method includes: monitoring the homocysteine concentration in blood of the individual; administering a composition including the compound to inhibit DYRK1A activity or the candidate compound; and assessing that the activity of the DYRK1A protein is suppressed by the administration of the composition in a case where the homocysteine concentration in blood is raised after the administration. In one or a plurality of embodiments, the individual is a living body, and the examples include a human being, a mouse, a rat and any other animal expressing DYRK1A protein.

In one or a plurality of embodiments, the present disclosure relates to a method of prevention, improvement, suppression of progression and/or treatment of Alzheimer's disease, including administration of a composition for inducing instability in an in vivo or intracellular DYRK1A protein or for reducing the amount of an in vivo or intracellular DYRK1A protein, and conducting a method of assessing activity of DYRK1A protein in an individual according to the present disclosure or conducting an assessment on the effect of administration of a composition including a compound to inhibit the DYRK1A activity or a candidate compound thereof.

### Examples

The present disclosure will be described below more specifically by referring to the following Examples, though the Examples are not intended to limit the present disclosure. It should be noted that the entire contents of the documents cited in the present disclosure are incorporated herein by reference.

### [Screening System of Compound to Reduce Phosphoenzyme DYRK1A Protein]

### [Production of Assay Cell Having Simultaneous Expression System for a Target Protein and an Internal Standard Protein]

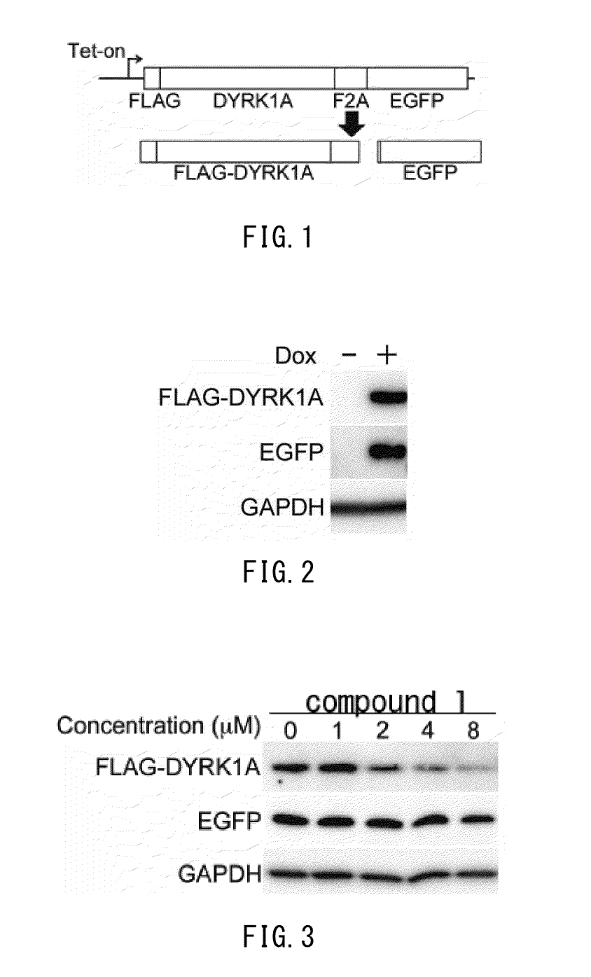

An assay cell having a simultaneous expression system of FLAG-tagged DYRK1A and EGFP (FLAG-DYRK1A-2A-EGFP) as shown in the model diagram of FIG. 1 was produced. Specifically, the cell was produced in the following manner. A FLAG tag was fused with DYRK1A (FLAG-DYRK1A), which was further connected in-frame by using 2A peptide and EGFP gene that codes a green-fluorescent protein (FLAG-DYRK1A-2A-EGFP). The 2A peptide is an amino acid sequence that enables bicistronic gene expression. Thereby, the FLAG-DYRK1A-2A-EGFP is simultaneously translated from the top of a single mRNA. A vector expressing the gene (FLAG-DYRK1A-2A-EGFP) was produced in the following manner. Namely, respective DNA components constituting the vector were isolated as DNA fragments from separate vectors by PCR. The respective fragments were tied sequentially by using an overlap elongation PCR and DNA ligation so as to construct an object vector. Lipofection was used for introduction into HEK293 cells derived from human embryonic nephrocyte. Since hygromycin resistance gene was integrated in advance into the object vector, by culturing the vector-introduced cells in the presence of hygromycin, only the cells where the vector was integrated stably in the chromosome were selected.

Within the thus produced assay cell, by addition of doxycycline (Dox), the FLAG-tagged DYRK1A and EGFP controlled by a Tet-on system were expression-induced. The results are shown in FIG. 2. FIG. 2 includes an example of Western blotting showing that FLAG-DYRK1A and EGFP are expression-induced by doxycycline.

### [Compound Screening by Use of Assay Cell]

Cultured cells with expressed FLAG-DYRK1A-2A-EGFP (assay cells) were cultured on a plate, and a test compound of a constant concentration was added to the culture solution for a further cultivation. After the cultivation, the cells were anchored to be subjected to a fluorescence cyto-staining by using an anti-FLAG tag antibody and an anti-EGFP antibody. The stained cell samples were introduced into an analyzer equipped with a fluorescence microscope so as to analyze quantitatively the amount of the anti-FLAG antibody and the amount of the anti-EGFP antibody, and the ratio was analyzed. From the analytical data, test compounds that change the ratio in comparison with a case of absence of such a test compound were selected as candidate compounds. One example thereof is shown in FIG. 3. FIG. 3 shows an example of Western blot analysis to indicate that the test compound (Compound 1 below) does not affect the amount of the internal standard EGFP protein but that it reduces only the amount of the FLAG-DYRK1A protein within the cell.

The obtained group of candidate compounds was used to review the respective concentration dependences and peculiarities, thereby obtaining the Compound 1 below.

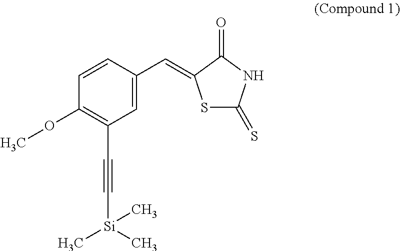 [SMILES: COc1ccc(/C=C2\SC(=S)NC2=O)cc1C#C[Si](C)(C)C]

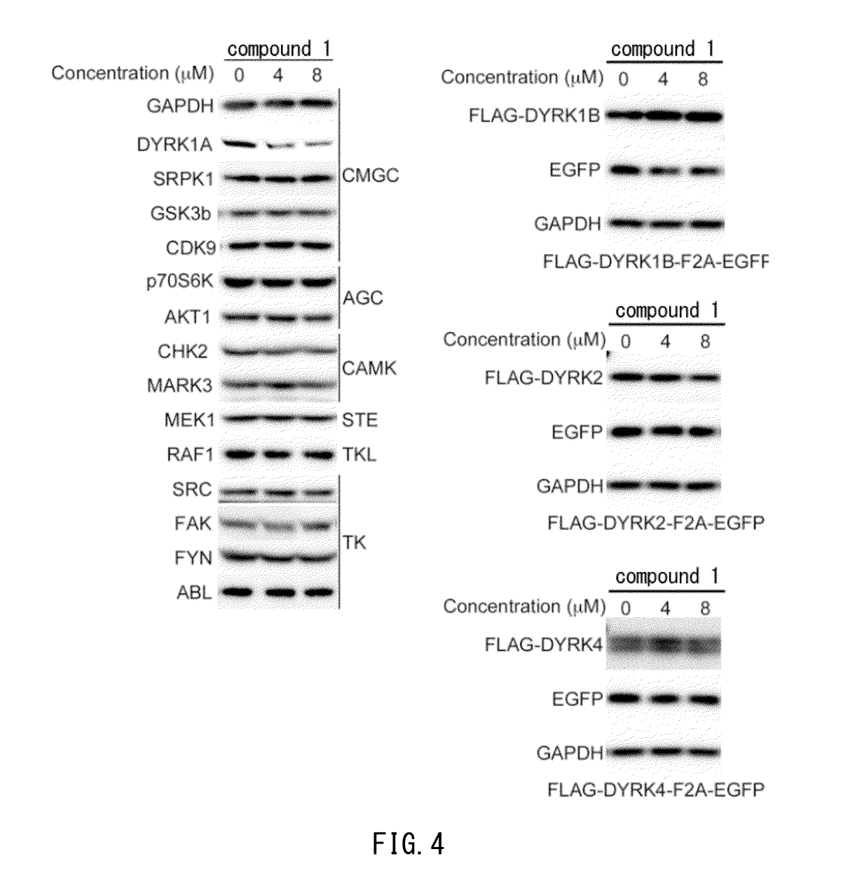

The Compound 1 had an activity not to affect at all transcription and translation of DYRK1A but to make DYRK1A protein unstable and allow the protein to decompose. Further, the Compound 1 did not exhibit an effect of making various phosphoenzymes (including DYRK1B, DYRK2 and DYRK4 as analogous phosphoenzymes) unstable (i.e., effect of reducing the amount of protein), but it exhibited a high peculiarity with respect to DYRK1A. One example thereof is shown in FIG. 4. FIG. 4 shows an example of a result of Western blot analysis of the protein amounts of various phosphoenzymes at the time of adding 0, 4 and 8 μm of the Compound 1. The Compound 1 exhibited a high peculiarity with respect to DYRK1A similarly with regard to a phosphorylation activity inhabitation effect.

### Production Example 1

### Production of Compound 1

The Compound 1 was produced in the following manner.

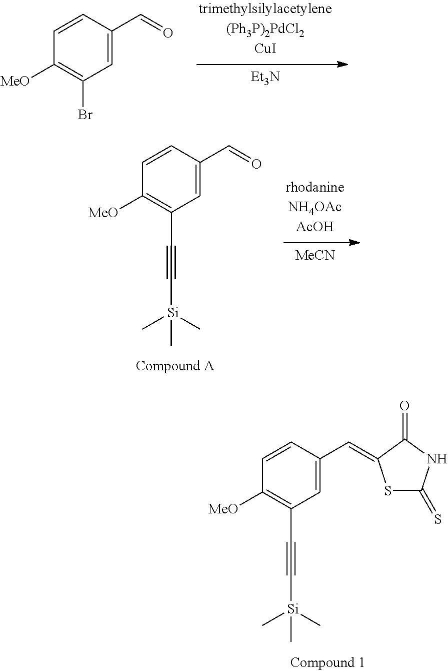 [SMILES: "Et3N" ; "MeCN" ; COc1ccc(C=O)cc1Br ; COc1ccc(C=O)cc1C#C[Si](C)(C)C ; COc1ccc(/C=C2\SC(=S)NC2=O)cc1C#C[Si](C)(C)C]

### [Synthesis of Compound A]

Under the argon atmosphere, trimethylsilylacetylene (5.5 mL, 40 mmol, commercially available product) was added at room temperature to a triethylamine (Et₃N) (100 mL) solution of 3-bromo-4-methoxybenzaldehyde (5.00 g, 23.3 mmol, commercially available product), dichlorobistriphenylphosphinepalladium ((Ph₃P)₂PdCl₂) (816 mg, 1.16 mmol, commercially available product) and copper iodide (CuI) (133 mg, 0.70 mmol, commercially available product), and the mixture was heated to reflux for 3 hours. Water was added thereto and the mixture was extracted three times by use of ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=5/1), thereby obtaining 4-methoxy-3-[2-(trimethylsilyl)ethynyl]benzaldehyde (Compound A) (4.24 g, 18.2 mmol, 78.1%) as a light-yellow solid.

TLC R_{f} 0.30 (n-hexane/ethyl acetate=5/1)

mp 55-56° C.

¹H NMR (CDCl₃, 500 MHz) δ 0.27 (s, 9H, Si(CH₃)₃), 3.96 (s, 3H, OCH₃), 6.98 (d, J=8.5 Hz, 1H, aromatic), 7.82 (d, J=8.5 Hz, 1H, aromatic), 7.97 (s, 1H aromatic), 9.85 (s, 1H, CHO)

¹³C NMR (CDCl₃, 126 MHz) δ−0.1, 56.3, 99.5, 100.1, 110.7, 113.3, 129.4, 131.8, 136.1, 164.7, 190.1

IR (cm⁻¹) 762, 849, 1125, 1263, 1499, 1591, 1690, 2155, 2965

### [Synthesis of Compound 1]

Under the argon atmosphere, acetic acid (AcOH) (0.5 mL, 8.62 mmol, commercially available product) was added at room temperature to an acetonitrile (MeCN) (50 mL) solution of Compound A (2.01 g, 8.62 mmol), ammonium acetate (NH₄OAc) (331 mg, 4.30 mmol, commercially available product) and rhodanine (1.15 g, 8.62 mmol, commercially available product), and the mixture was heated to reflux for 3 hours. After allowing the mixture to cool to room temperature, water (H₂O) (10 mL) was added thereto, and a precipitated solid was filtered with a Hirsch funnel, and then washed on the funnel three times with water and twice with diethyl ether sequentially, thereby obtaining ((Z)-5-(4-methoxy-3-((trimethylsilyl)ethynyl)benzylidene)-2-thioxothiazolidin-4-one) (Compound 1) (2.64 g, 7.60 mmol, 88.2%) as a yellow solid.

mp 215-216° C.

¹H NMR (DMSO-d₆, 500 MHz) δ 0.24 (s, 9H, Si(CH₃)₃), 3.90 (s, 3H, OCH₃), 7.24 (d, J=8.5 Hz, 1H, aromatic), 7.59-7.64 (m, 3H, aromatic and olefinic), 13.81 (brs, 1H, NH) ¹³C NMR (DMSO-d₆, 126 MHz) δ−0.1, 56.3, 99.3, 100.4, 112.3, 112.5, 123.5, 125.5, 130.8, 132.7, 136.1, 161.6, 169.4, 195.3

IR (cm⁻¹) 849, 1275, 1433, 1499, 1586, 1692, 2839, 2895, 2943, 2970, 3038, 3057, 3152

### [Compound Screening System for Reducing Neurodegeneration Disease-Related Protein TAU]

### [Preparation of Assay Cell Having Simultaneous Expression System of Target Protein and Internal Standard Protein]

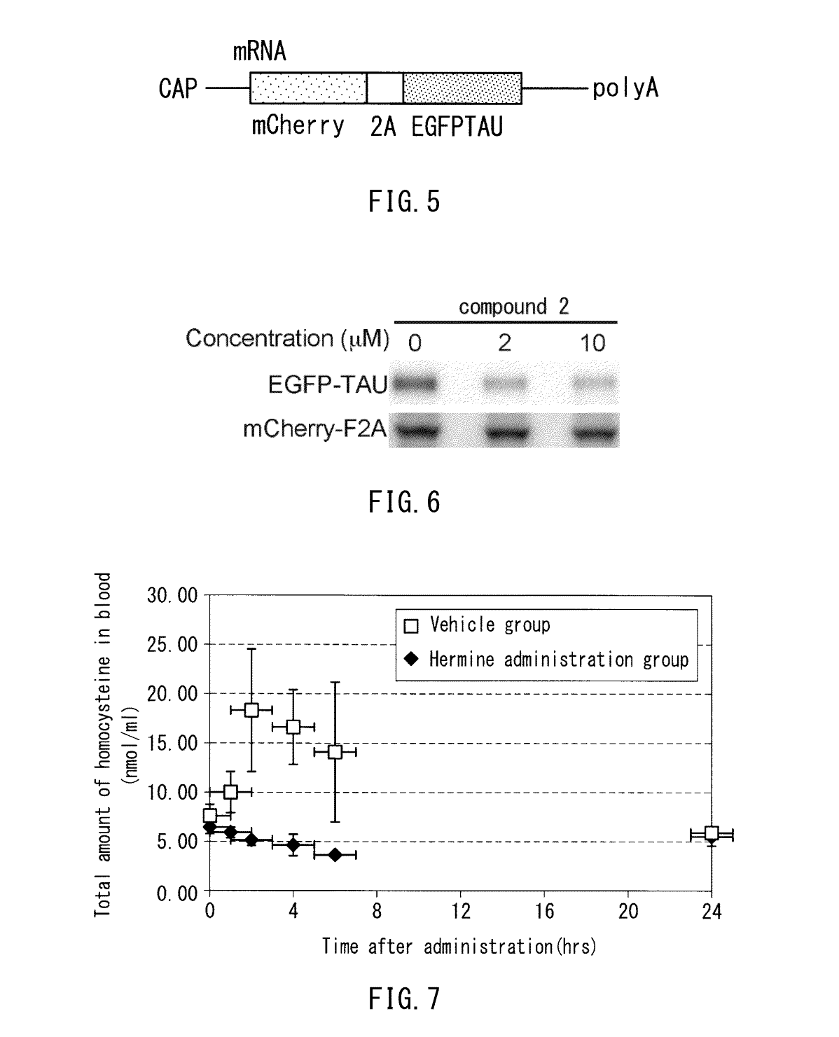

An assay cell having the simultaneous expression system of EGFP-fused TAU and mCherry (mCherry-2A-EGFP-TAU) as shown in the model diagram of FIG. 5 was produced. Specifically, the process was as follows. EGFP was fused with TAU (EGFP-TAU), and further, it was connected in-frame by using 2A peptide and mCherry gene that codes a red fluorescent protein (mCherry-2A-EGFP-TAU). The 2A peptide is an amino acid sequence enabling a bicistronic gene expression. Thereby, mCherry-2A-EGFP-TAU is translated simultaneously from the top of a single mRNA. A vector expressing the gene (mCherry-2A-EGFP-TAU) was produced in the following manner. Namely, respective DNA components constituting the vector were isolated as DNA fragments from separate vectors by PCR. The respective fragments were tied sequentially by using an overlap elongation PCR and DNA ligation so as to construct an object vector. Lipofection was used for introduction into HEK293 cells derived from human embryonic nephrocyte. Since hygromycin resistance gene was integrated in advance into the object vector, by culturing the vector-introduced cells in the presence of hygromycin, only the cells where the vector was integrated stably in the chromosome were selected.

### [Compound Screening by Use of Assay Cell]

Cultured cells with expressed mCherry-2A-EGFP-TAU (assay cells) were cultured on a plate and a test compound of a constant concentration was added to the culture solution for a further cultivation. After the cultivation, the cells were introduced into an analyzer equipped with a fluorescence microscope so as to analyze quantitatively the amount of EGFP and the amount of mCherry, and the ratio was analyzed. From the analytical data, test compounds that change the ratio in comparison with a case of absence of such a test compound were selected as candidate compounds. One example thereof is shown in FIG. 6. FIG. 6 shows an example of Western blot analysis to indicate that the test compound (Compound 2 below) does not affect the amount of the internal standard mCherry protein but that it reduces only the amount of the EGFP-TAU protein within the cells.

The obtained group of candidate compounds was used to review the respective concentration dependences and peculiarities, thereby obtaining the Compound 2 below. The Compound 2 had an activity not to affect at all transcription and translation of TAU but to make TAU protein unstable and allow the protein to decompose.

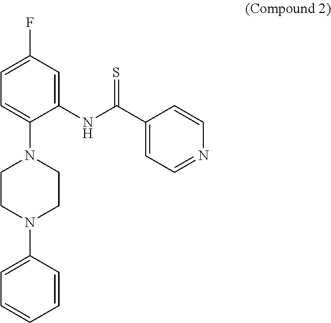 [SMILES: Fc1ccc(N2CCN(c3ccccc3)CC2)c(NC(=S)c2ccncc2)c1]

### Production Example 2

### Production of Compound 2

The Compound 2 was produced in the following manner.

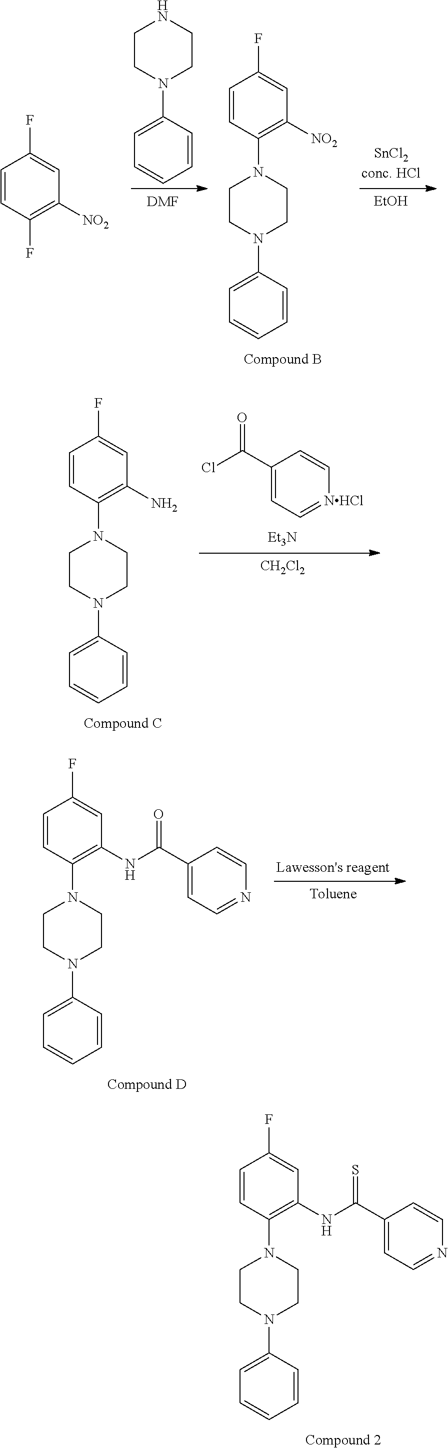

### [Synthesis of Compound B]

To N,N-dimethyl formamide (DMF) (20 mL) solution of 1,4-difluoro-2-nitrobenzene (2.00 g, 12.6 mmol, commercially available product), 1-phenylpiperazine (6.11 g, 37.7 mmol, commercially available product) was added at room temperature, and the mixture was stirred for 13 hours. Water was added thereto, and the mixture was extracted three times by use of ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=7/1), thereby obtaining 1-(4-fluoro-2-nitrophenyl)-4-phenylpiperazine (Compound B) (3.82 g, 12.6 mmol, quant.) as an orange-colored oily material.

TLC R_{f} 0.30 (n-hexane/ethyl acetate=7/1)

### [Synthesis of Compound C]

Concentrated hydrochloric acid (6.86 mL, 82.4 mmol) and anhydrous stannic dichloride (7.21 g, 38.0 mmol) were added sequentially at 0° C. to an ethanol (40 mL) solution of Compound B (3.82 g, 12.6 mmol). The temperature was raised again to room temperature and the mixture was stirred for 2 hours. To this, a saturated aqueous solution of sodium hydrogencarbonate was added and the mixture was extracted three times by using ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=19/1), thereby obtaining 2-(4-phenyl-1-piperazinyl)-5-fluoroaniline (Compound C) (<3.69 g, <13.6 mmol, quant.) as a colorless oily material.

TLC R_{f} 0.33 (n-hexane/ethyl acetate=5/1)

mp 161-163° C.

¹H NMR (CDCl₃, 500 MHz) δ 3.02 (t, J=4.5 Hz, 4H, 2CH₂), 3.20-3.45 (br, 4H, 2CH₂), 4.16 (s, 2H, NH₂), 6.42 (dd, J=2.5, 8.5 Hz, 1H, aromatic), 6.45 (dd, J=2.5, 10.5 Hz, 1H, aromatic), 6.89 (t, J=7.5 Hz, 1H, aromatic), 6.96-7.02 (m, 3H, aromatic), 7.28-7.32 (m, 2H, aromatic)

¹³C NMR (CDCl₃, 126 MHz) δ 50.3, 51.8, 101.0 (d, J=26.5 Hz), 104.5 (d, J=22.7 Hz), 116.5 (d, J=31.5 Hz), 120.1, 121.2 (d, J=10.1 Hz), 129.4 (d, J=3.8 Hz), 135.2 (d, J=2.5 Hz), 143.4 (d, J=11.3 Hz), 151.5, 160.7 (d, J=241 Hz)

¹⁹F NMR (CDCl₃, 376 MHz) δ −113.5-−113.4 (m)

IR (cm⁻¹) 546, 698, 768, 802, 837, 935, 972, 1142, 1159, 1175, 1207, 1229, 1260, 1290, 1310, 1377, 1450, 1493, 1504, 1576, 1599, 1612, 2835, 3354, 3453

### [Synthesis of Compound D]

Isonicotnic acid chloride hydrochloride (980 mg, 5.52 mmol, commercially available product) and triethylamine (1.15 mL, 8.29 mmol) were added sequentially at 0° C. to a dichloromethane (15 mL) solution of Compound C (500 mg, 1.84 mmol). The temperature was raised again to room temperature and the mixture was stirred for 13 hours. Water was added thereto, and the mixture was extracted three times by use of ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=2/1), thereby obtaining N-[5-fluoro-2-(4-phenyl-1-piperazinyl)phenyl]isonicotinamide (Compound D) (556 mg, 1.48 mmol, 80.4%) as a colorless solid.

TLC R_{f} 0.47 (n-hexane/ethyl acetate=1/1)

mp 203-204° C.

¹H NMR, (CDCl₃, 500 MHz) δ 3.08 (t, J=4.5 Hz, 4H, 2CH₂), 3.20-3.54 (br, 4H, 2CH₂), 6.86 (ddd, J=3.0, 8.5, 8.5 Hz, 1H, aromatic), 6.94 (t, J=7.0 Hz, 1H), 7.01 (dd, J=1.0, 9.0 Hz, 2H, aromatic), 7.27 (dd, J=5.5, 8.5 Hz, 1H, aromatic), 7.31-7.36 (m, 2H, aromatic), 7.74 (AA′BB′, 2H, aromatic), 8.39 (dd, 1H, J=3.0, 10.5 Hz, 1H, aromatic), 8.83 (AA′BB′, 2H, aromatic), 9.76 (s, 1H, NH)

¹³C NMR (CDCl₃, 126 MHz) δ 50.6, 53.1, 107.3 (d, J=29.0 Hz), 111.1 (d, J=22.7 Hz), 116.5, 120.7, 120.8, 122.5 (d, J=10.1 Hz), 129.5, 134.7 (d, J=12.6 Hz), 137.3 (d, J=3.8 Hz), 141.8, 151.1, 151.2 160M (d, J=244 Hz), 162.9

¹⁹F NMR (CDCl₃, 376 MHz) δ−118.2-−118.1 (m)

IR (cm⁻¹) 692, 748, 768, 939, 1140, 1159, 1231, 1269, 1377, 1445, 1495, 1533, 1557, 1605, 1678, 2828, 3273

### [Synthesis of Compound 2]

Lawesson's reagent (294 mg, 0.729 mmol, commercially available product) was added to a toluene (30 mL) solution of Compound D (183 mg, 0.486 mmol), and the mixture was reflux-stirred at 130° C. for 20 hours. After lowering the temperature to room temperature, the mixture was purified by silica gel column chromatography (n-hexane/ethyl acetate=2/1), thereby obtaining N-[5-fluoro-2-(4-phenyl-1-piperazinyl)phenyl]isonicotinthioamide (Compound 2) (122 mg, 0.311 mmol, 64.0%) as a yellow solid.

TLC R_{f} 0.50 (n-hexane/ethyl acetate=1/1)

mp 183-186° C.

¹H NMR (CDCl₃, 500 MHz) δ 3.08 (t, J=4.5 Hz, 4H, 2CH₂), 3.20-3.40 (br, 4H, 2CH₂), 6.91-7.03 (m, 4H, aromatic), 7.27-7.34 (m, 3H, aromatic), 7.71 (d, J=5.5 Hz, 2H, aromatic), 8.73 (d, J=5.5 Hz, 2H, aromatic), 9.28 (d, 11.5 Hz, 1H, aromatic), 11.0 (s, 1H, NH)

¹³C NMR (CDCl₃, 126 MHz) δ 50.3, 53.0, 107.5 (d, J=30.2 Hz), 112.9 (d, J=22.7 Hz), 116.4, 120.1, 120.7, 122.3 (d, J=8.8 Hz), 129.3, 135.7 (d, J=11.3 Hz), 138.8 (d, J=2.5 Hz), 149.3, 150.6, 150.7, 159.6 (d, J=244 Hz), 192.2

¹⁹F NMR (CDCl₃, 376 MHz) δ−112.4-−112.6 (m)

IR (cm⁻¹) 733, 760, 818, 937, 1155, 1229, 1263, 1314, 1364, 1377, 1410, 1449, 1495, 1518, 1599, 2826

### [Monitoring of DYRK1A Activity Inhibition Using Homocysteine in Blood as Index]

DYRK1A inhibitor Harmine was orally administered to rats, and the post-administration homocysteine concentration in blood was measured. The specific conditions are as follows, and the results are illustrated in FIG. 7.

After dissolving the DYRK1A inhibitor Harmine in an administration solvent of 0.9% NaCl, about 1 ml per individual was orally administered (final administration concentration: 18 mg/kg). At certain interval after the administration, the blood was collected from caudate veins. From the blood, blood plasma was separated and preserved in the presence of EDTA-2Na. Analysis of the homocysteine concentration in the blood plasma was consigned to SRL. Specifically, the measurement was conducted by extracting fractures including homocysteine from the blood plasma and then analyzing the amount of homocysteine included in the extract by HPLC (see Araki A et al: Journal of Chromatography 422, 43-52 1987, Araki A: Gendai-Iryou 22, 10, 2544-2549 1990).

As shown in FIG. 7, after two hours from the oral administration, the homocysteine in blood was raised rapidly, and it exhibited higher numerical values in comparison with controls for 5 hours. Therefore, the homocysteine in blood can be an index to illustrate the in vivo inhibition activity of a DYRK1A inhibitor or in vivo DYRK1A activity.

### [Screening System 2 of Compound for Reducing DYRK1A Protein]

### [Compound Screening by Use of Assay Cell]

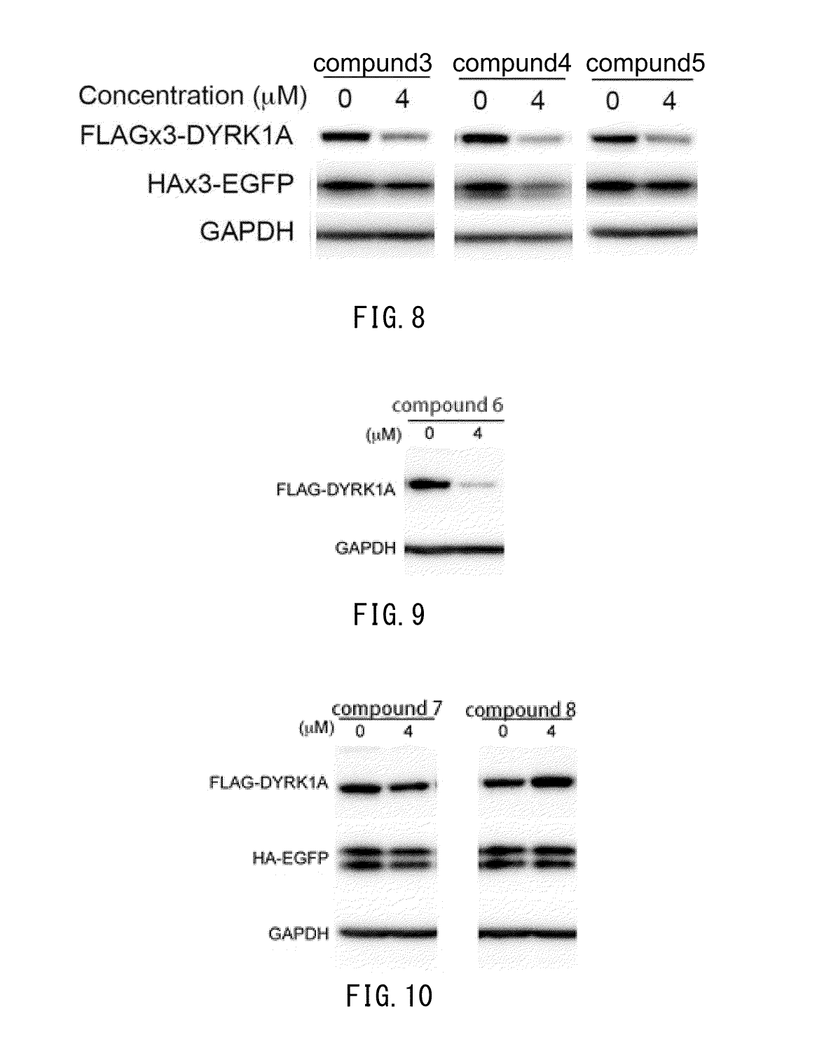

Cultured cells with expressed FLAGx3-DYRK1A-2A-HAx3-EGFP (assay cells) were cultured on a plate, and test compounds of a constant concentration were added to the culture solution for a further cultivation. After the cultivation, the amounts of FLAGx3-DYRK1A, HAx3-EGFP, and GAPDH were quantitatively analyzed by a Western blotting method, and the ratio was analyzed. From the analytical data, test compounds that change the ratio in comparison with a case of absence of such a test compound were selected as candidate compounds. One example thereof is shown in FIG. 8. FIG. 8 shows examples of Western blot analysis to indicate that the test compounds (Compounds 3, 4 and 5) reduce FLAGx3-DYRK1A protein within the cells. It was found that the Compounds 3, 4 and 5 at concentration of 4 μm were capable of reducing the amount of intracellular DYRK1A protein as shown in FIG. 8.

### Production Example 3

### Production of Compound 3

The Compound 3 was produced in the following manner.

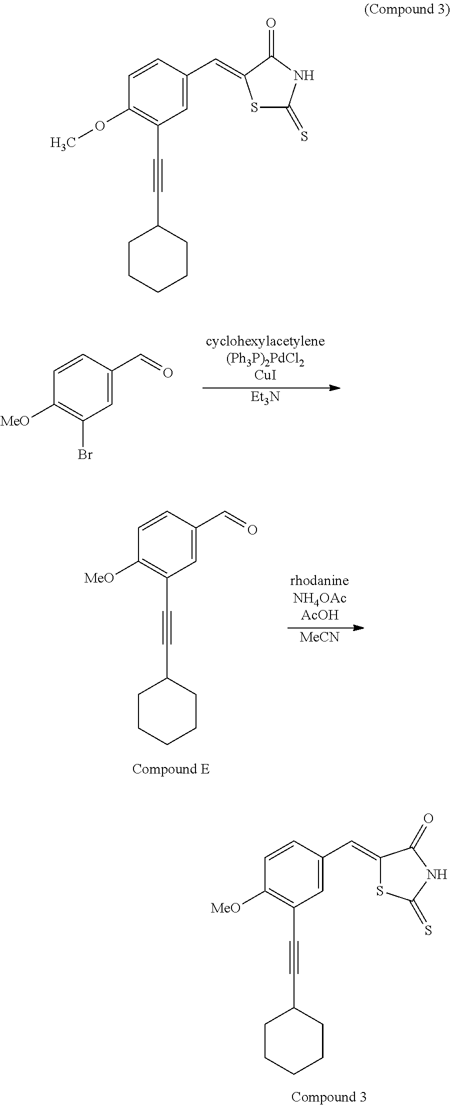 [SMILES: "Et3N" ; "MeCN" ; COc1ccc(/C=C2\SC(=S)NC2=O)cc1C#CC1CCCCC1 ; COc1ccc(C=O)cc1Br ; COc1ccc(C=O)cc1C#CC1CCCCC1 ; COc1ccc(/C=C2\SC(=S)NC2=O)cc1C#CC1CCCCC1]

### [Synthesis of Compound E]

Under the argon atmosphere, cyclohexylacetylene (260 μL, 2.00 mmol, commercially available product) was added at room temperature to a triethylamine (Et₃N) (2 mL) solution of 3-bromo-4-methoxybenzaldehyde (215 mg, 1.00 mmol, commercially available product), dichlorobistriphenylphosphinepalladium ((Ph₃P)₂PdCl₂) (35.1 mg, 50.0 μmol, commercially available product) and copper iodide (CuI) (5.7 mg, 30.0 μmol, commercially available product), and the mixture was heated to reflux for 8 hours. Water was added thereto and the mixture was extracted three times by use of ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=5/1), thereby obtaining 3-(cyclohexylethynyl)-4-methoxybenzaldehyde (Compound E) (211 mg, 870 μmol, 87.0%) as a brown oily material.

TLC R_{f} 0.25 (n-hexane/ethyl acetate=5/1)

¹H NMR (CDCl₃, 500 MHz) δ 1.32-1.41 (m, 3H, cyclohexyl), 1.50-1.62 (m, 3H, cyclohexyl), 1.73-1.80 (m, 2H, cyclohexyl), 1.89-1.91 (m, 2H, cyclohexyl), 2.63-2.68 (m, 1H, CH), 3.95 (s, 3H, OCH₃), 6.96 (d, J=8.5 Hz, 1H, aromatic), 7.77 (dd, J=2.5, 8.5 Hz, 1H, aromatic), 7.90 (d, J=2.5 Hz, 1H, aromatic), 9.84 (s, 1H, CHO)

¹³C NMR (CDCl₃, 126 MHz) δ 24.8, 25.9, 29.9, 32.6, 56.2, 75.3, 100.1, 110.5, 114.3, 129.5, 130.9, 135.5, 164.4, 190.4

IR (cm⁻¹) 768, 816, 1020, 1593, 1697, 2228, 2853, 2930, 3503

### [Synthesis of Compound 3]

Under the argon atmosphere, acetic acid (AcOH) (57 μL, 1.00 mmol, commercially available product) was added at room temperature to an acetonitrile (MeCN) (2 mL) solution of Compound E (242 mg, 1.00 mmol), ammonium acetate (NH₄OAc) (38.5 mg, 500 μmol, commercially available product), and rhodanine (133 mg, 1.00 mmol, commercially available product), and the mixture was heated to reflux for 2 hours. After allowing the mixture to cool to room temperature, water (H₂O) (1 mL) was added thereto, and a precipitated solid was filtered with a Hirsch funnel, and then washed on the funnel three times with water and twice with diethyl ether sequentially, thereby obtaining (Z)-5-(3-(cyclohexylethynyl)-4-methoxybenzylidene)-2-thioxothiazolidin-4-one (Compound 3) (207 mg, 579 μmol, 57.9%) as a yellow solid.

mp 185-186° C.

¹H NMR (DMSO-d₆, 500 MHz) δ 1.34-1.36 (m, 3H, cyclohexyl), 1.46-1.50 (m, 3H, cyclohexyl), 1.68-1.71 (m, 2H, cyclohexyl), 1.79-1.81 (m, 2H, cyclohexyl), 2.65-2.69 (m, 1H, CH), 3.87 (s, 3H, OCH₃), 7.20 (d, J=9.5 Hz, 1H, aromatic), 7.53-7.58 (m, 3H, aromatic and olefinic), 13.79 (brs, 1H, NH)

¹³C NMR (DMSO-d₆, 126 MHz) δ 24.6, 25.8, 29.4, 32.5, 56.6, 76.4, 99.8, 112.7, 113.9, 123.5, 125.8, 131.5, 132.3, 135.8, 161.7, 169.8, 195.8

IR (cm⁻¹), 675, 1148, 1586, 1695, 2849, 2899, 2926, 3036, 3050, 3146

### Production Example 4

### Production of Compound 4

The Compound 4 was produced in the following manner.

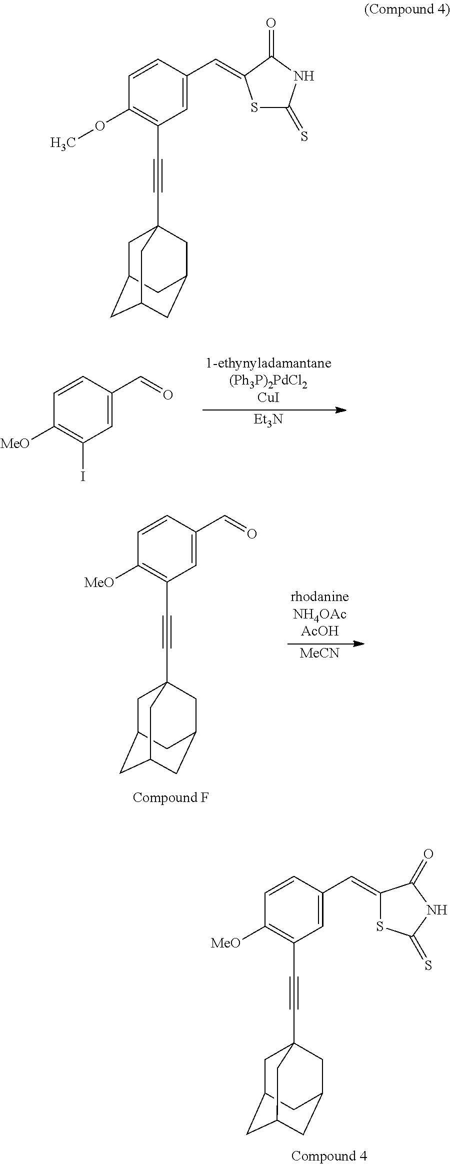 [SMILES: "Et3N" ; "MeCN" ; COc1ccc(/C=C2\SC(=S)NC2=O)cc1C#CC12CC3CC(CC(C3)C1)C2 ; COc1ccc(C=O)cc1I ; COc1ccc(C=O)cc1C#CC12CC3CC(CC(C3)C1)C2 ; COc1ccc(/C=C2\SC(=S)NC2=O)cc1C#CC12CC3CC(CC(C3)C1)C2]

### [Synthesis of Compound F]

Under the argon atmosphere, 1-ethynyladamantane (192 mg, 1.20 mmol, commercially available product) was added at room temperature to a triethylamine (Et₃N) (2 mL) solution of 3-iodo-4-methoxybenzaldehyde (262 mg, 1.00 mmol, commercially available product), dichlorobistriphenylphosphinepalladium ((Ph₃P)₂PdCl₂) (35.1 mg, 50.0 μmol, commercially available product) and copper iodide (CuI) (5.7 mg, 30 μmol, commercially available product), and the mixture was heated to reflux for 10 hours. Water was added thereto and the mixture was extracted three times by use of ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=5/1), thereby obtaining 3-(1-adamantylethynyl)-4-methoxybenzaldehyde (Compound F) (172 mg, 584 μmol, 58.4%) as a brown solid.

mp 86-87° C.

TLC R_{f} 0.35 (n-hexane/ethyl acetate=5/1)

¹H NMR (CDCl₃, 500 MHz) δ 1.72 (brs, 6H, adamantyl), 1.98 (brs, 9H, adamantyl), 3.93 (s, 3H, OCH₃), 6.93 (d, J=8.5 Hz, 1H, aromatic), 7.75 (dd, J=2.0, 8.5 Hz, 1H, aromatic), 7.88 (d, J=2.0 Hz, 1H, aromatic), 9.83 (s, 1H, CHO)

¹³C NMR (CDCl₃, 126 MHz) δ 27.9, 30.3, 36.3, 42.7, 56.2, 74.0, 104.0, 110.5, 114.2, 129.4, 130.8, 135.5, 164.3, 190.4

IR (cm⁻¹) 814, 1138, 1271, 1501, 1684, 2359, 2849, 2903

### [Synthesis of Compound 4]

Under the argon atmosphere, acetic acid (AcOH) (33 μL, 580 μmol, commercially available product) was added at room temperature to an acetonitrile (MeCN) (2 mL) solution of Compound F (172 mg, 580 μmol), ammonium acetate (NH₄OAc) (22.4 mg, 290 μmol, commercially available product) and rhodanine (77.3 mg, 580 μmol, commercially available product), and the mixture was heated to reflux for 3 hours. After allowing the mixture to cool to room temperature, water (H₂O) (1 mL) was added thereto, and a precipitated solid was filtered with a Hirsch funnel, and then washed on the funnel three times with water and twice with diethyl ether sequentially, thereby obtaining (Z)-5-(3-(2-adamantylethynyl)-4-methoxybenzylidene)-2-thioxothiazolidin-4-one (Compound 4) (148 mg, 361 μmol, 62.2%) as a yellow solid.

mp 266-267° C.

¹H NMR (DMSO-d₆, 500 MHz) δ 1.68 (brs, 6H, adamantyl), 1.90 (brs, 6H, adamantyl), 1.95 (brs, 3H, adamantyl), 3.86 (s, 3H, OCH₃), 7.18 (d, J=8.5 Hz, 1H, aromatic), 7.50 (s, 1H, aromatic), 7.53 (d, J=8.5 Hz, 1H, aromatic), 7.57 (s, 1H, olefinic), 13.78 (brs, 1H, CHO)

¹³C NMR (DMSO-d₆, 126 MHz) δ 27.3, 29.9, 35.7, 42.3, 56.2, 74.6, 103.1, 112.3, 113.4, 123.0, 125.4, 131.1, 131.8, 135.4, 161.1, 169.3, 195.3

IR (cm⁻¹) 667, 801, 1190, 1425, 1501, 1589, 1703, 2847, 2903, 2928, 3061, 3069, 3154

### Production Example 5

### Production of Compound 5

The Compound 5 was produced in the following manner.

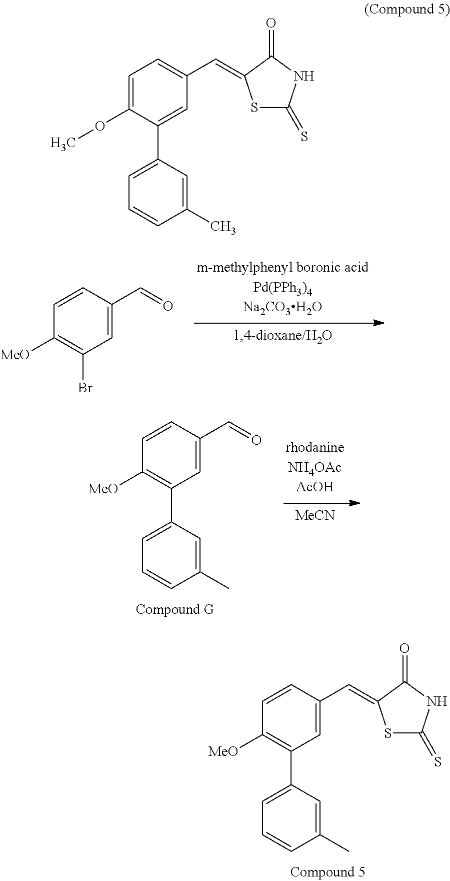 [SMILES: "MeCN" ; COc1ccc(/C=C2\SC(=S)NC2=O)cc1-c1cccc(C)c1 ; COc1ccc(C=O)cc1Br ; "1,4-dioxane/H2O" ; COc1ccc(C=O)cc1-c1cccc(C)c1 ; COc1ccc(/C=C2\SC(=S)NC2=O)cc1-c1cccc(C)c1]

### [Synthesis of Compound G]

Under the argon atmosphere, water (2 mL) was added at room temperature to a 1,4-dioxane (Et₃N) (15 mL) solution of 3-bromo-4-methoxybenzaldehyde (215 mg, 1.00 mmol, commercially available product), m-methylphenyl boronic acid (163 mg, 1.20 mmol), tetrakistriphenylphosphinepalladium (Pd(PPh₃)₄) (57.8 mg, 50.0 μmol, commercially available product) and sodium carbonate monohydrate (Na₂CO₃.H₂O) (248 mg, 2.00 mmol, commercially available product), and the mixture was heated to reflux for 7.5 hours. Water was added thereto and the mixture was extracted three times by use of ethyl acetate. The obtained organic layer was washed with saturated saline, dried over anhydrous sodium sulfate, filtered and then concentrated under a reduced pressure. The residue was purified by silica gel column chromatography (n-hexane/ethyl acetate=5/1), thereby obtaining 3-(3′-methylphenyl)-4-methoxybenzaldehyde (Compound G) (224 mg, 990 μmol, 99.0%) as a light-yellow oily material.

TLC R_{f} 0.30 (n-hexane/ethyl acetate=5/1)

¹H NMR (CDCl₃, 500 MHz) δ 2.43 (s, 3H, CH₃), 3.91 (s, 3H, OCH₃), 7.10 (d, J=9.0 Hz, 1H, aromatic), 7.19-7.21 (m, 1H, aromatic), 7.34-7.52 (m, 3H, aromatic), 7.86-7.89 (m, 2H, aromatic), 9.94 (s, 1H, CHO)

¹³C NMR (CDCl₃, 126 MHz) δ 21.4, 55.8, 110.9, 126.5, 127.9, 128.3, 129.7, 130.0, 131.2, 131.4, 132.2, 136.9, 137.7, 161.4, 190.9

IR (cm⁻¹) 702, 791, 1022, 1202, 1370, 1499, 1595, 1686, 2837, 2943

### [Synthesis of Compound 5]

Under the argon atmosphere, acetic acid (AcOH) (57 μL, 1.0 mmol, commercially available product) was added at room temperature to an acetonitrile (MeCN) (2 mL) solution of Compound G (226 mg, 1.00 mmol), ammonium acetate (NH₄OAc) (38.5 mg, 500 μmol, commercially available product) and rhodanine (133 mg, 1.00 mmol, commercially available product), and the mixture was heated to reflux for 3 hours. After allowing the mixture to cool to room temperature, water (H₂O) (1 mL) was added thereto, and a precipitated solid was filtered with a Hirsch funnel, and then washed on the funnel three times with water and twice with diethyl ether sequentially, thereby obtaining (Z)-5-(3-(3′-methylphenyl))-4-methoxybenzylidene)-2-thioxothiazolidin-4-one (Compound 5) (311 mg, 911 μmol, 91.1%) as an orange-colored solid.

mp 204-205° C.

¹H NMR (DMSO-d₆, 500 MHz) δ 2.33 (s, 3H, CH₃), 3.93 (s, 3H, OCH₃), 7.23-7.27 (m, 3H, aromatic), 7.43 (s, 1H, aromatic), 7.44 (s, 1H, aromatic), 7.57-7.69 (m, 2H, aromatic), 7.70 (s, 1H, olefinic), 13.80 (brs, 1H, NH)

¹³C NMR (DMSO-d₆, 126 MHz) δ 21.1, 56.3, 84.3, 94.1, 99.5, 112.4, 112.6, 119.2, 125.8, 129.4 (2C), 131.3 (2C), 132.5, 135.2, 138.7, 161.0, 196.1

IR (cm⁻¹) 679, 1018, 1200, 1582, 1701, 2839, 2909, 2943, 2963, 3017, 3036, 3050, 3148

### [Screening System 3 of Compound for Reducing or Varying DYRK1A Protein]

### [Compound Screening by Use of Assay Cell]

Cultured cells with expressed FLAGx3-DYRK1A-2A-HAx3-EGFP (assay cells) were cultured on a plate and a test compound of a constant concentration was added to the culture solution for a further cultivation. After the cultivation, the amounts of FLAGx3-DYRK1A, HAx3-EGFP, and GAPDH were quantitatively analyzed by a Western blotting method, and the ratio was analyzed. From the analytical data, test compounds that change the ratio in comparison with a case of absence of such a test compound were selected as candidate compounds. Examples thereof are shown in FIGS. 9 and 10. FIG. 9 shows an example of Western blot analysis to indicate that the test compound (Compound 6) reduces FLAGx3-DYRK1A protein within the cells. Further, the left of FIG. 10 shows an example of Western blot analysis to indicate that the test compound (Compound 7) does not affect the amount of the internal standard EGFP protein but reduces only the amount of the FLAG-DYRK1A protein within the cells. Further, the right of FIG. 10 shows an example of Western blot analysis to indicate that the test compound (Compound 8) does not affect the amount of the internal standard EGFP protein but increases only the amount of the FLAG-DYRK1A protein within the cells.

### Production Example 6

### Production of Compound 6

The Compound 6 was produced in the following manner.

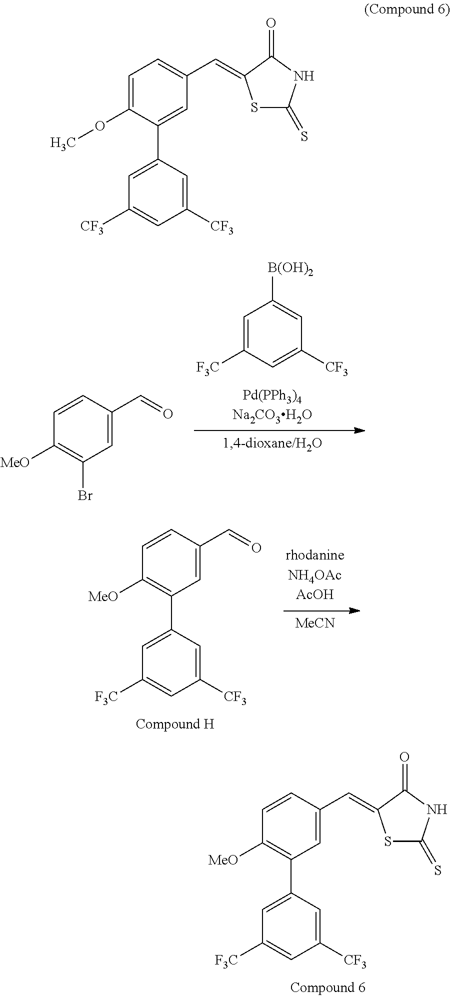

### [Synthesis of Compound H]

Under the argon atmosphere, water (H₂O) (2 mL) was added at room temperature to a 1,4-dioxane (Et₃N) (15 mL) solution of 3-bromo-4-methoxybenzaldehyde (215 mg, 1.00 mmol, commercially available product), 3,5-bis(trifluoromethyl)phenylboronic acid (310 mg, 1.20 mmol, commercially available product), tetrakistriphenylphosphinepalladium (Pd(PPh₃)₄) (57.8 mg, 50.0 μmol, commercially available product) and sodium carbonate monohydrate (Na₂CO₃.H₂O) (248 mg, 2.00 mmol, commercially available product), and the mixture was heated to reflux for 22 hours. Water was added thereto and the mixture was extracted three times by use of ethyl acetate. The obtained organic layer was washed with saturated saline, dried over anhydrous sodium sulfate, filtered and then concentrated under a reduced pressure, thereby obtaining 3,5-bis(trifluoromethyl)phenyl-4-methoxybenzaldehyde (Compound H) as a brown oily material. This was used for the next reaction, without purification.

### [Synthesis of Compound 6]

Under the argon atmosphere, acetic acid (AcOH) (57 μL, 1.0 mmol, commercially available product) was added at room temperature to an acetonitrile (MeCN) (2 mL) solution of Compound H (3,5-bis(trifluoromethyl)phenyl-4-methoxybenzaldehyde) (<1 mmol), ammonium acetate (NH₄OAc) (38.5 mg, 0.500 mmol, commercially available product) and rhodanine (133 mg, 1.00 mmol, commercially available product), and the mixture was heated to reflux for 3 hours. After allowing the mixture to cool to room temperature, water (H₂O) (10 mL) was added thereto, and a precipitated crystal was filtered with a Hirsch funnel, and then washed three times with water and twice with diethyl ether, thereby obtaining (Z)-5-(3,5-bis(trifluoromethyl)phenyl-4-methoxybenzylidene)-2-thioxothiazolidin-4-one (Compound 6) (236 mg, 0.509 mmol, 50.9%) as a yellow solid.

mp 244-245° C.

¹H NMR, (CDCl₃, 400 MHz) δ 3.92 (s, 3H, OCH₃), 7.14 (d, J=8.8 Hz, 1H, aromatic), 7.44 (d, J=2.4 Hz, 1H, olefinic), 7.56 (dd, J=8.8, 2.4 Hz, 1H, aromatic), 7.66 (s, 1H, aromatic), 7.89 (s, 1H, aromatic), 7.95 (s, 2H, aromatic), 9.41 (brs, 1H, NH)

IR (cm⁻¹) 686, 808, 1154, 1436, 1594, 1699, 3191, 3445

### Production Example 7

### Production of Compound 7

The Compound 7 was produced in the following manner.

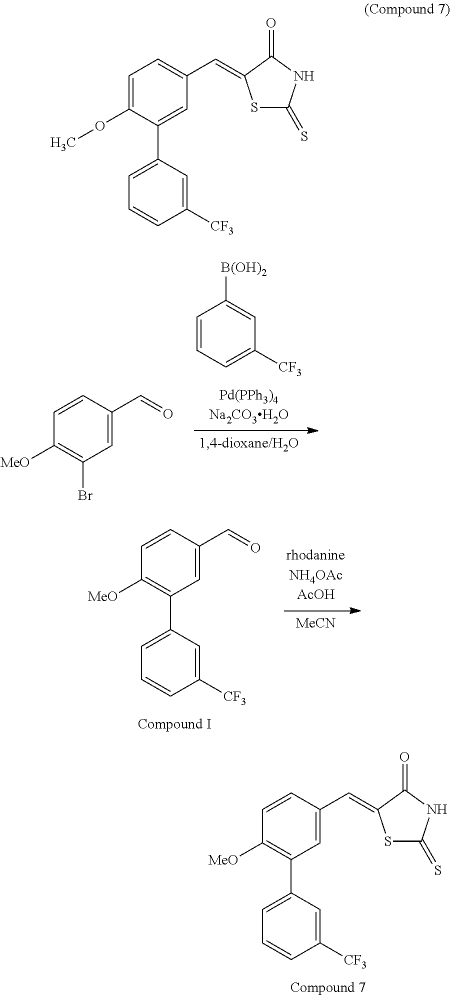

### [Synthesis of Compound I]

Under the argon atmosphere, water (2 mL) was added at room temperature to a 1,4-dioxane (Et₃N) (15 mL) solution of 3-bromo-4-methoxybenzaldehyde (215 mg, 1.00 mmol, commercially available product), 3-(trifluoromethyl)phenylboronic acid (228 mg, 1.20 mmol, commercially available product) tetrakistriphenylphosphinepalladium (Pd(PPh₃)₄) (57.8 mg, 50.0 μmol, commercially available product), sodium carbonate monohydrate (Na₂CO₃.H₂O) (248 mg, 2.00 mmol, commercially available product), and the mixture was heated to reflux for 22 hours. Water was added thereto and the mixture was extracted three times by use of ethyl acetate. The obtained organic layer was washed with saturated saline, dried over anhydrous sodium sulfate, filtered and then concentrated under a reduced pressure, thereby obtaining 3-(trifluoromethyl)phenyl-4-methoxybenzaldehyde (Compound I) as a yellow oily material. This was used for the next reaction, without purification.

### [Synthesis of Compound 7]

Under the argon atmosphere, acetic acid (AcOH) (57 μL, 1.0 mmol, commercially available product) was added at room temperature to an acetonitrile (MeCN) (2 mL) solution of Compound I (3-(trifluoromethyl)phenyl-4-methoxybenzaldehyde) (<1 mmol), ammonium acetate (NH₄OAc) (38.5 mg, 0.500 mmol, commercially available product) and rhodanine (133 mg, 1.00 mmol, commercially available product), and the mixture was heated to reflux for 3 hours. After radiationally cooling to room temperature, water (H₂O) (10 mL) was added thereto, and a precipitated crystal was filtered with a Hirsch funnel, and then washed three times with water and twice with diethyl ether, thereby obtaining (Z)-5-(3-(trifluoromethyl)phenyl-4-methoxybenzylidene)-2-thioxothiazolidin-4-one (Compound 7) (127 mg, 0.321 mmol, 32.1%) as a yellow solid.

mp 209-210° C.

¹H NMR (CDCl₃, 400 MHz) δ 3.91 (s, 3H, OCH₃), 7.11 (d, J=8.4 Hz, 1H, aromatic), 7.44 (d, J=2.0 Hz, 1H, olefinic), 7.52 (dd, J=8.4, 2.0 Hz, 1H, aromatic), 7.58 (d, J=7.6 Hz, 1H, aromatic), 7.69-7.63 (m, 3H, aromatic), 7.78 (s, 2H, aromatic), 9.22 (brs, 1H, NH)

IR (cm⁻¹) 555, 696, 806, 1023, 1272, 1339, 1570, 1689, 3049, 3153

### Production Example 8

### Production of Compound 8

The Compound 8 was produced in the following manner.

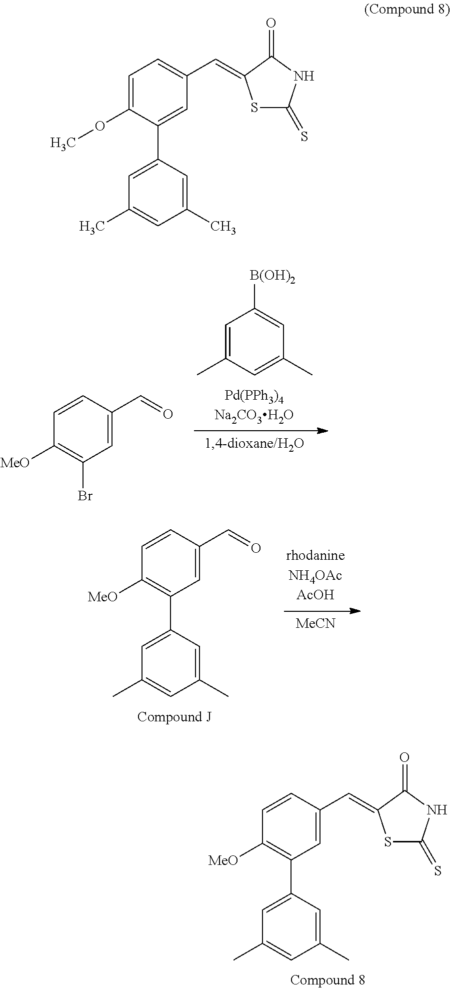

### [Synthesis of Compound J]

Under the argon atmosphere, water (H₂O) (2 mL) was added at room temperature to a 1,4-dioxane (Et₃N) (15 mL) solution of 3-bromo-4-methoxybenzaldehyde (215 mg, 1.00 mmol, commercially available product), 3,5-dimethylphenylboronic acid (228 mg, 1.20 mmol, commercially available product), tetrakistriphenylphosphinepalladium (Pd(PPh₃)₄) (57.8 mg, 50.0 μmol, commercially available product) and sodium carbonate monohydrate (Na₂CO₃.H₂O) (248 mg, 2.00 mmol, commercially available product), and the mixture was heated to reflux for 22 hours. Water was added thereto and the mixture was extracted three times by use of ethyl acetate. The obtained organic layer was washed with saturated saline, dried over anhydrous sodium sulfate, filtered and then concentrated under a reduced pressure, thereby obtaining 3,5-dimethylphenyl-4-methoxybenzaldehyde (Compound J) as a yellow oily material. This was used for the next reaction, without purification.

### [Synthesis of Compound 8]

Under the argon atmosphere, acetic acid (AcOH) (57 μL, 1.0 mmol, commercially available product) was added at room temperature to an acetonitrile (MeCN) (2 mL) solution of Compound J (3,5-dimethylphenyl-4-methoxybenzaldehyde) (<1 mmol), ammonium acetate (NH₄OAc) (38.5 mg, 0.500 mmol, commercially available product) and rhodanine (133 mg, 1.00 mmol, commercially available product), and the mixture was heated to reflux for 3 hours. After allowing the mixture to cool to room temperature, water (H₂O) (10 mL) was added thereto, and a precipitated crystal was filtered with a Hirsch funnel, and then washed three times with water and twice with diethyl ether, thereby obtaining (Z)-5-(3,5-dimethylphenyl-4-methoxybenzylidene)-2-thioxothiazolidin-4-one (Compound 8) (309 mg, 0.870 mmol, 87.0%) as a yellow solid. mp 238-239° C.

¹H NMR (CDCl₃, 400 MHz) δ 2.38 (s, 6H, CH₃), 3.89 (s, 3H, OCH₃), 7.03 (s, aromatic), 7.07 (d, J=8.4 Hz, 1H, aromatic), 7.10 (s, 1H, aromatic), 7.42 (d, J=2.4 Hz, 1H, olefinic), 7.47 (dd, J=8.4, 2.4 Hz, 1H, aromatic), 7.67 (s, 1H, aromatic), 9.30 (brs, 1H, NH)

IR (cm⁻¹) 701, 801, 849, 1025, 1286, 1397, 1568, 1686, 2870, 3047, 3143

### [Screening System 4 of Compound for Reducing DYRK1A Protein]

### [Compound Screening by Use of Assay Cell]

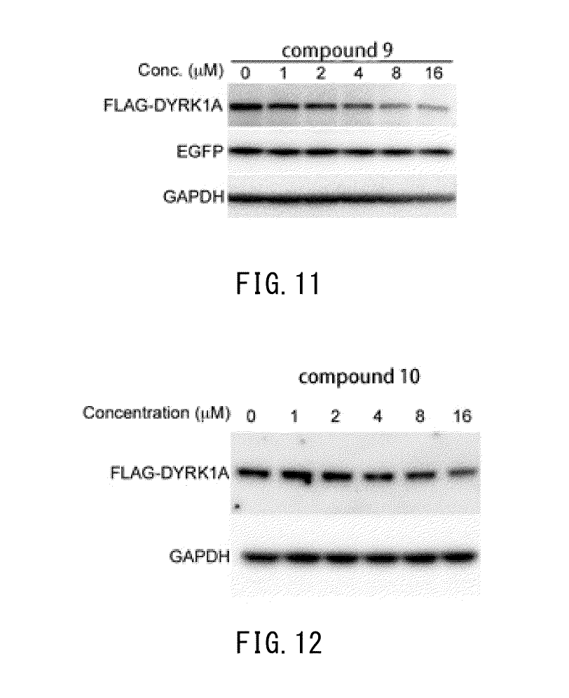

Cultured cells with expressed FLAGx3-DYRK1A-2A-HAx3-EGFP (assay cells) were cultured on a plate, and a test compound of a constant concentration was added to the culture solution for a further cultivation. After the cultivation, the respective amounts of FLAGx3-DYRK1A, HAx3-EGFP, and GAPDH were quantitatively analyzed by a Western blotting method, and the ratio was analyzed. From the analytical data, test compounds that change the ratio in comparison with a case of absence of such a test compound were selected as candidate compounds. Examples thereof are shown in FIGS. 11 and 12. FIG. 11 shows an example of Western blot analysis to indicate that the test compound (Compound 9) does not affect the amount of the internal standard EGFP protein but that it reduces only the amount of the FLAG-DYRK1A protein within the cells. FIG. 12 shows an example of Western blot analysis to indicate that the test compound (Compound 10) reduces the FLAGx3-DYRK1A protein within the cells.

### Production Example 9

### Production of Compound 9

The Compound 9 was produced in the following manner.

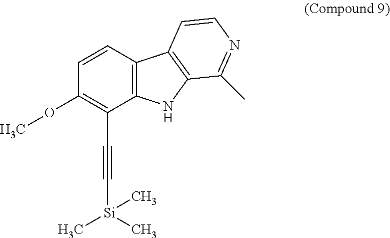

### [Synthesis of Compound 9]

Under the argon atmosphere, trimethylsilylacetylene (55 μL, 0.40 mmol, commercially available product) was added at room temperature to a toluene (dehydrate, 2.0 mL)-triethylamine (Et₃N) (2.0 mL) solution of 8-iodoharmine (67.6 mg, 0.200 mmol, synthetic compound (US2007027199A1)), dichlorobistriphenylphosphinepalladium ((Ph₃P)₂PdCl₂) (7.0 mg, 10 μmol, commercially available product), copper iodide (CuI) (3.8 mg, 20 μmol, commercially available product) and triphenylphosphine (PPh₃) (5.2 mg, 20 μmol, commercially available product), and the mixture was heated and stirred at 60° C. for 4 hours. After allowing the mixture to cool to room temperature, a saturated aqueous solution of ammonium chloride was added thereto, and the mixture was extracted three times by use of ethyl acetate. The obtained organic layer was dried over anhydrous sodium sulfate, filtered and then concentrated under a reduced pressure. The residue was purified by silica gel column chromatography (ethyl acetate), thereby obtaining 8-[2-(trimethylsilyl)ethynyl]harmine (Compound 9) (35.8 mg, 0.116 mmol, 58.0%) as a colorless solid.

TLC R_{f} 0.40 (ethyl acetate).

mp 185-186° C.

¹H NMR (CDCl₃, 500 MHz) δ 0.37 (s, 9H, Si(CH₃)₃), 2.83 (s, 3H, CH₃), 4.02 (s, 3H, OCH₃), 6.87 (d, J=8.5 Hz, 1H, aromatic), 7.70 (d, J=5.0 Hz, 1H, aromatic), 7.98 (d, J=8.5 Hz, 1H, aromatic), 8.12 (brs, 1H, NH), 8.35 (brs, 1H, aromatic)

¹³C NMR (CDCl₃, 126 MHz) δ 0.3 (3C), 20.2, 56.6, 94.8, 96.9, 104.3, 104.7, 112.4, 115.8, 123.2, 128.9, 134.4, 139.5, 141.3, 142.9, 161.0

IR (cm⁻¹) 775, 945, 1099, 1339, 1450, 1614, 2146, 2767, 2861, 2977

### Production Example 10

### Production of Compound 10

The Compound 10 was produced in the following manner.

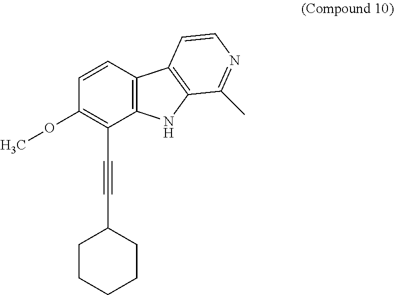

### [Synthesis of Compound 10]

Under the argon atmosphere, cyclohexylacetylene (26 μL, 0.20 mmol, commercially available product) was added at room temperature to a toluene (dehydrate, 1.0 mL)-triethylamine (Et₃N) (1.0 mL) solution of 8-iodoharmine (33.8 mg, 0.100 mmol, synthetic compound (US2007027199A1)), dichlorobistriphenylphosphinepalladium ((Ph₃P)₂PdCl₂) (3.5 mg, 5.0 μmol, commercially available product), copper iodide (Cup (1.9 mg, 10 μmol, commercially available product) and triphenylphosphine (PPh₃) (2.6 mg, 0.20 mmol, commercially available product), and the mixture was heated and stirred at 60° C. for 11 hours. After allowing the mixture to cool to room temperature, a saturated aqueous solution of ammonium chloride was added thereto, and the mixture was extracted three times by use of ethyl acetate. The obtained organic layer was dried over anhydrous sodium sulfate, filtered and then concentrated under a reduced pressure. The residue was purified by silica gel column chromatography (ethyl acetate), thereby obtaining 8-[2-(cyclohexyl)ethynyl]harmine (Compound 10) (30.3 mg, 95.2 μmol, 95.2%) as a colorless solid.

TLC R_{F} 0.30 (ethyl acetate)

MP 198-199° C.

¹H NMR (CDCL₃, 500 MHZ) δ 1.68 (BRS, 4H, —CH₂—×2), 1.44 (BRS, 2H, —CH₂—), 1.84 (BRS, 2H, —CH₂—), 2.00 (BRS, 2H, —CH₂—), 2.85-2.80 (M, 1H, —CH—), 2.82 (S, 3H, CH₃), 4.00 (S, 3H, OCH₃), 6.88 (D, J=8.5 HZ, 1H, AROMATIC), 7.70 (D, J=5.0 HZ, 1H, AROMATIC), 7.93 (D, J=8.5 HZ, 1H, AROMATIC), 8.12 (BRS, 1H, NH), 8.33 (D, J=5.0 HZ, 1H, AROMATIC)

¹³C NMR (CDCL₃, 126 MHZ) δ 20.2 (2C), 24.9, 25.9, 30.3, 32.9 (2C), 56.7, 72.3, 95.6, 104.0, 105.0, 112.4, 115.7, 121.9, 129.0, 134.4, 139.3, 141.2, 142.7, 160.4

IR (CM⁻¹) 792, 1088, 1230, 1245, 1288, 1423, 1448, 1616, 2851, 2930

1-21. (canceled)

22. A composition for inducing instability in an in vivo or intracellular TAU protein or for reducing the amount of an in vivo or intracellular TAU protein, comprising a compound represented by General formula (II) below or a pharmaceutically acceptable salt thereof: 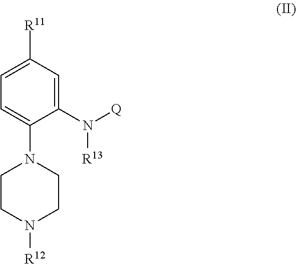
[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

23-24. (canceled)

25. A method for inducing instability in an in vivo or intracellular TAU protein, or for reducing the amount of an in vivo or intracellular TAU protein, the method comprising administration of the compound represented by General formula (II) below or the pharmaceutically acceptable salt thereof to either a living body or a cell: 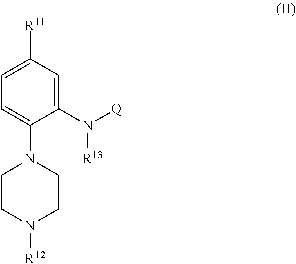
[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

26. The method according to claim 25, comprising administration of a compound represented by: 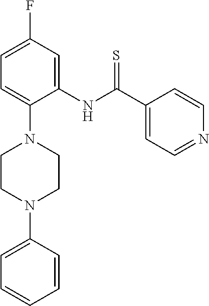 [SMILES: Fc1ccc(N2CCN(c3ccccc3)CC2)c(NC(=S)c2ccncc2)c1]
or a pharmaceutically acceptable salt thereof to a living body or a cell.

27. A pharmaceutical composition for prevention, improvement, suppression of progression, and/or treatment of Alzheimer's disease and/or Tauopathies, containing a compound represented by General formula (II) below or the pharmaceutically acceptable salt thereof as an active ingredient: 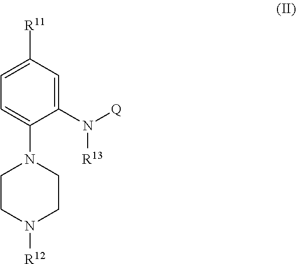
[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

28-29. (canceled)

30. A method for prevention, improvement, suppression of progression, and/or treatment of Alzheimer's disease and/or Tauopathies, the method comprising administration of a compound represented by General formula (II) or a pharmaceutically acceptable salt thereof to a subject: 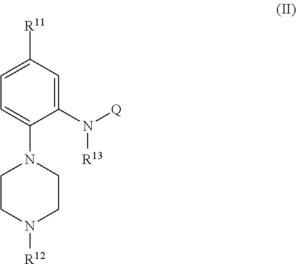
[In Formula (II), R¹¹ is a halogen atom or a C₁₋₆ alkyl group that may be substituted with a halogen atom; R¹² is a hydrogen atom, a C₁₋₆ alkyl group, or a phenyl group or a monocyclic heteroaromatic group unsubstituted or substituted with a halogen atom; R¹³ is a hydrogen atom or a C₁₋₆ alkyl group; Q is a group selected from the group consisting of —C(O/S)—C═C—R¹⁴, —C(O/S)—NH—CH₂—R¹⁴, —C(O/S)—NH—C(O/S)—R¹⁴, —C(O/S)—R¹⁴ and —SO₂—R¹⁴; R¹⁴ is a phenyl group unsubstituted or substituted with a C₁₋₆ alkyl group, a C₁₋₆ alkoxy group, a hydroxyl group or a halogen atom, or a monocyclic heteroaromatic group.]

31. The method according to claim 25, comprising administration of a compound represented by: 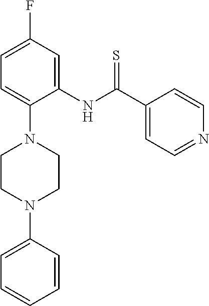 [SMILES: Fc1ccc(N2CCN(c3ccccc3)CC2)c(NC(=S)c2ccncc2)c1]
or a pharmaceutically acceptable salt thereof to a living body or a cell.

32-49. (canceled)
